(19) United States
(12) Patent Application Publication
(10) Pub. No.: US 2023/0139671 A1
(43) Pub. Date: May 4, 2023

(54) OSMOTIC MEDIATED RELEASE SYNTHETIC NANOCARRIERS

(71) Applicant: Selecta Biosciences, Inc., Watertown, MA (US)

(72) Inventors: David H. Altreuter, Wayland, MA (US); Aaron P. Griset, Somerville, MA (US)

(73) Assignee: Selecta Biosciences, Inc., Watertown, MA (US)

(21) Appl. No.: 17/815,877

(22) Filed: Jul. 28, 2022

Related U.S. Application Data
(62) Division of application No. 13/428,340, filed on Mar. 23, 2012, now abandoned.
(60) Provisional application No. 61/467,595, filed on Mar. 25, 2011.

(51) Int. Cl. A61K 9/00; A61K 9/51; A61K 9/19; C12N 15/88

(52) U.S. Cl. CPC A61K 9/0004; A61K 9/19; A61K 9/5146; A61K 9/5153; A61K 9/5192; C12N 15/88; A61K 2039/60

(57) ABSTRACT

This invention relates, at least in part, to osmotic mediated release barrier-free synthetic nanocarriers and methods of production and use.

### RELATED APPLICATIONS

This application claims the benefit under 35 U.S.5 C. §119 of United States provisional application 61/467,595, filed Mar. 25, 2011, the entire contents of which are incorporated herein by reference.

### FIELD OF THE INVENTION

This invention relates, at least in part, to osmotic mediated release barrier-free synthetic nanocarriers and methods of production and use.

### BACKGROUND OF THE INVENTION

Safe and effective delivery to patients of osmotically active agents, such as isolated nucleic acids or isolated peptides, is a current therapeutic limitation. Liposomes, microparticles, nanoparticles, polymersomes, solid-lipid-particles, and the like have been utilized in an attempt to provide for delivery of osmotically active agents. Many of these systems conventionally utilize positively-charged surfactants or polymers and/or a durable diffusion-impermeable barrier to secure the osmotically active agent to/within the carrier. These systems tend to be limited in their utility because of potential toxicity of the cationic elements and/or by low rates of release of the osmotically active agent from the system. The low rates of release may be attributed to the cationic agent, the relatively low % w/w loading of the system, or the nature of the diffusive barrier.

Therefore, what is needed are compositions and methods that address the problems in the art as noted above.

### SUMMARY OF THE INVENTION

In one aspect, a dosage form comprising osmotic mediated release barrier-free synthetic nanocarriers comprising an encapsulated osmotically active agent is provided. In one embodiment, the dosage form further comprises a vehicle having an osmolality of 200-500 mOsm/kg. In one embodiment, the osmotic mediated release barrier-free synthetic nanocarriers comprise pH triggered osmotic mediated release barrier-free synthetic nanocarriers.

In another aspect, a method comprising forming osmotic mediated release barrier-free synthetic nanocarriers that comprise an osmotically active agent in an environment having an osmolality ranging from 200-500 mOsm/kg; and maintaining the formed osmotic mediated release barrier-free synthetic nanocarriers in an environment having an osmolality ranging from 200-500 mOsm/kg is provided. In one embodiment, the environment in which the osmotic mediated release barrier-free synthetic nanocarriers are formed, and the environment in which the osmotic mediated release barrier-free synthetic nanocarriers are maintained, are the same. In one embodiment, the method further comprises processing the formed osmotic mediated release barrier-free synthetic nanocarriers in an environment having an osmolality ranging from 200-500 mOsm/kg. In one embodiment, the processing comprises: washing the synthetic nanocarriers, centrifuging the synthetic nanocarriers, filtering the synthetic nanocarriers, concentrating or diluting the synthetic nanocarriers, freezing the synthetic nanocarriers, drying the synthetic nanocarriers, combining the synthetic nanocarriers with other synthetic nanocarriers or with additive agents or excipients, adjusting the pH or buffer environment of the synthetic nanocarriers, entrapping the synthetic nanocarriers in a gel or high-viscosity medium, resuspending the synthetic nanocarriers, surface modifying the synthetic nanocarriers covalently or by physical processes such as coating or annealing, impregnating or doping the synthetic nanocarriers with active agents or excipients, sterilizing the synthetic nanocarriers, reconstituting the synthetic nanocarriers for administration, or combinations of any of the above. In one embodiment, the method further comprises storing the formed osmotic mediated release barrier-free synthetic nanocarriers in an environment having an osmolality ranging from 200-500 mOsm/kg. In one embodiment, the method further comprises formulating the formed osmotic mediated release barrier-free synthetic nanocarriers into a dosage form that maintains the formed osmotic mediated release barrier-free synthetic nanocarriers in an environment having an osmolality ranging from 200-500 mOsm/kg.

In another aspect, a process for producing a dosage form comprising osmotic mediated release barrier-free synthetic nanocarriers comprising the method steps as defined in any of the methods provided is provided.

In another aspect, a dosage form comprising any of the osmotic mediated release barrier-free synthetic nanocarriers is provided. Such synthetic nanocarriers may be made according to any of the methods or processes provided. Such synthetic nanocarriers may be produced or obtainable by any of the methods or processes provided.

In another aspect, a lyophilized dosage form comprising lyophilized osmotic mediated release barrier-free synthetic nanocarriers comprising an encapsulated osmotically active agent; and lyophilizing agents that provide a vehicle having an osmolality of 200-500 mOsm/kg upon reconstitution of the lyophilized dosage form is provided. In one embodiment, the lyophilizing agents comprise comprise salts and buffering agents, simple or complex carbohydrates, polyols, pH adjustment agents, chelating and antioxidant agents, stabilizers and preservatives, or surfactants. In one embodiment, the salts and buffering agents comprise NaCl, NaPO₄, or Tris, the simple or complex carbohydrates comprise sucrose, dextrose, dextran, or carboxymethyl cellulose, the polyols comprise mannitol, sorbitol, glycerol, or polyvinyl alcohol, the pH adjustment agents comprise HCl, NaOH, or sodium citrate, the chelating and antioxidant agents comprise EDTA, ascorbic acid, or alpha-tocopherol, the stabilizers and preservatives comprise gelatin, glycine, histidine, or benzyl alcohol, and/or the surfactants comprise polysorbate 80, sodium deoxycholate, or Triton X-100. In one embodiment, the osmotic mediated release barrier-free synthetic nanocarriers comprise pH triggered osmotic mediated release barrier-free synthetic nanocarriers.

In another aspect, a method comprising providing osmotic mediated release barrier-free synthetic nanocarriers that comprise an osmotically active agent in an environment having an osmolality ranging from 200-500 mOsm/kg; and administering the osmotic mediated release barrier-free synthetic nanocarriers to a subject is provided. In one embodiment, the method further comprises processing the formed osmotic mediated release barrier-free synthetic nanocarriers only in environments having an osmolality ranging from 200-500 mOsm/kg. In one embodiment, the processing comprises: washing the synthetic nanocarriers, centrifuging the synthetic nanocarriers, filtering the synthetic nanocarriers, concentrating or diluting the synthetic nanocarriers, freezing the synthetic nanocarriers, drying the synthetic nanocarriers, combining the synthetic nanocarriers with other synthetic nanocarriers or with additive agents or excipients, adjusting the pH or buffer environment of the synthetic nanocarriers, entrapping the synthetic nanocarriers in a gel or high-viscosity medium, resuspending the synthetic nanocarriers, surface modifying the synthetic nanocarriers covalently or by physical processes such as coating or annealing, impregnating or doping the synthetic nanocarriers with active agents or excipients, sterilizing the synthetic nanocarriers, reconstituting the synthetic nanocarriers for administration, or combinations of any of the above. In another embodiment, the method further comprises storing the formed osmotic mediated release barrier-free synthetic nanocarriers in an environment having an osmolality ranging from 200-500 mOsm/kg. In another embodiment, the method further comprises formulating the formed osmotic mediated release barrier-free synthetic nanocarriers into a dosage form that maintains the formed osmotic mediated release barrier-free synthetic nanocarriers in an environment having an osmolality ranging from 200-500 mOsm/kg.

In another aspect, a method of administering any of the compositions or dosage forms provided to a subject is provided. In one embodiment, the subject is in need thereof. In one embodiment, the subject has cancer, an infectious disease, a metabolic disease, a degenerative disease, an autoimmune disease, or an inflammatory disease. In one embodiment, the subject has an addiction. In one embodiment, the subject has been exposed to a toxin. In one embodiment, the composition or dosage form is in an amount effective to treat the subject.

In another aspect, a kit, comprising any of the compositions or dosage forms provided is provided. In one embodiment, the dosage form is a lyophilized dosage form. In one embodiment, the kit further comprises instructions for use and/or mixing. In one embodiment, the kit further comprises an agent for reconstitution or a pharmaceutically acceptable carrier.

In another aspect, any of the compositions or dosage forms provided may be for use in therapy or prophylaxis. In another aspect, any of the compositions or dosage forms provided may be for use in any of the methods provided. In another aspect, any of the compositions or dosage forms provided may be for use in a method of modulating, for example, inducing, enhancing, suppressing, directing, or redirecting, an immune response. In another aspect, any of the compositions or dosage forms provided may be for use in a method of treating or preventing cancer, an infectious disease, a metabolic disease, a degenerative disease, an autoimmune disease, an inflammatory disease, an immunological disease, an addiction, or a condition resulting from the exposure to a toxin, hazardous substance, environmental toxin, or other harmful agent. In another aspect, any of the compositions or dosage forms provided may be for use in a method of therapy or prophylaxis comprising administration by a subcutaneous, intramuscular, intradermal, oral, intranasal, transmucosal, sublingual, rectal, ophthalmic, transdermal, transcutaneous route or by a combination of these routes. In another aspect, use of any of the compositions or dosage forms provided for the manufacture of a medicament for use in any of the methods provided is provided.

In one embodiment, the osmotically active agent is present in the synthetic nanocarriers in an amount of about 2 weight percent, based on the total theoretical weight of the synthetic nanocarriers. In another embodiment, the osmotically active agent is present in the synthetic nanocarriers in an amount of about 3 weight percent, based on the total theoretical weight of the synthetic nanocarriers. In another embodiment, the osmotically active agent is present in the synthetic nanocarriers in an amount of about 4 weight percent, based on the total theoretical weight of the synthetic nanocarriers. In another embodiment, the osmotically active agent is present in the synthetic nanocarriers in an amount of about 5 weight percent, based on the total theoretical weight of the synthetic nanocarriers. In another embodiment, the osmotically active agent is present in the synthetic nanocarriers in an amount of about 6 weight percent, based on the total theoretical weight of the synthetic nanocarriers. In another embodiment, the osmotically active agent is present in the synthetic nanocarriers in an amount of about 7 weight percent, based on the total theoretical weight of the synthetic nanocarriers. In another embodiment, the osmotically active agent is present in the synthetic nanocarriers in an amount of about 8 weight percent, based on the total theoretical weight of the synthetic nanocarriers.

In one embodiment, the osmotically active agent comprises an isolated nucleic acid, a polymer, an isolated peptide, an isolated saccharide, macrocycle, or ions, cofactors, coenzymes, ligands, hydrophobically-paired agents, or hydrogen-bond donors or acceptors of any of the above. In one embodiment, the isolated nucleic acid comprises an immunostimulatory nucleic acid, immunostimulatory oligonucleotides, small interfering RNA, RNA interference oligonucleotides, RNA activating oligonucleotides, micro RNA oligonucleotides, antisense oligonucleotides, aptamers, gene therapy oligonucleotides, natural form plasmids, non-natural plasmids, chemically modified plasmids, chimeras that include oligonucleotide-based sequences, and combinations of any of the above. In another embodiment, the polymer comprises osmotically active dendrimers, polylactic acids, polyglycolic acids, poly lactic-co-glycolic acids, polycaprolactams, polyethylene glycols, polyacrylates, polymethacrylates, and co-polymers and/or combinations of any of the above. In another embodiment, the isolated peptide comprises osmotically active immunomodulatory peptides, MHC Class I or MHC Class II binding peptides, antigenic peptides, hormones and hormone mimetics, ligands, antibacterial and antimicrobial peptides, anti-coagulation peptides, and enzyme inhibitors. In another embodiment, the isolated saccharide comprises osmotically active antigenic saccharides, lipopolysaccharides, protein or peptide mimetic saccharides, cell surface targeting saccharides, anticoagulants, anti-inflammatory saccharides, anti-proliferative saccharides, including their natural and modified forms, monosaccharides, disaccharides, trisaccharides, oligosaccharides, or polysaccharides.

### BRIEF DESCRIPTION OF THE FIGURES

FIG. 1 demonstrates that oligonucleotide losses were driven by media osmolality for an already-formed and loaded nanocarrier.

FIG. 2 shows the percent release versus osmolality.

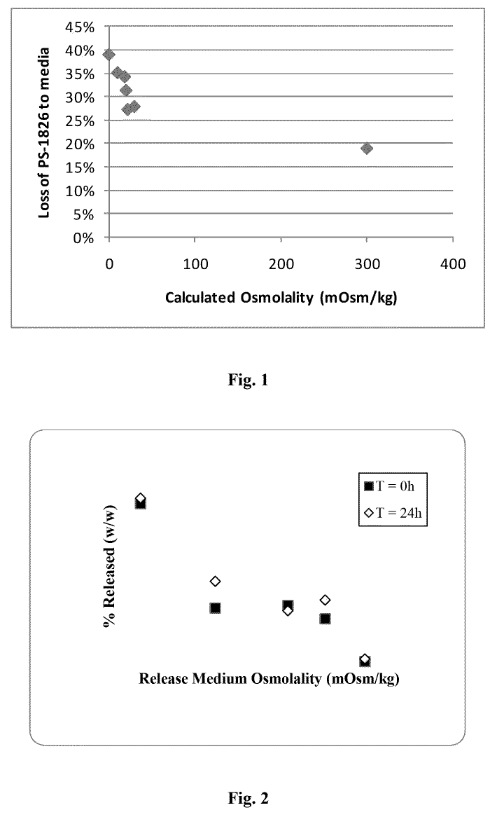

### DETAILED DESCRIPTION OF THE INVENTION

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

As used in this specification and the appended claims, the singular forms “a,” “an” and “the” include plural referents unless the content clearly dictates otherwise. For example, reference to “a polymer” includes a mixture of two or more such molecules or a mixture of differing molecular weights of a single polymer species, reference to “a synthetic nanocarrier” includes a mixture of two or more such synthetic nanocarriers or a plurality of such synthetic nanocarriers, reference to “a DNA molecule” includes a mixture of two or more such DNA molecules or a plurality of such DNA molecules, reference to “an adjuvant” includes a mixture of two or more such materials or a plurality of adjuvant molecules, and the like.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of ”or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

### A. Introduction

The inventors have unexpectedly and surprisingly discovered that the problems and limitations noted above can be overcome by practicing the invention disclosed herein. In particular, the inventors have unexpectedly discovered that it is possible to provide compositions, and related methods, that comprise dosage forms comprising osmotic mediated release barrier-free synthetic nanocarriers comprising an encapsulated osmotically active agent. The invention also relates to methods comprising: forming osmotic mediated release barrier-free synthetic nanocarriers that comprise an osmotically active agent in an environment having an osmolality ranging from 200-500 mOsm/kg; and maintaining the formed osmotic mediated release barrier-free synthetic nanocarriers in an environment having an osmolality ranging from 200-500 mOsm/kg. The invention further relates to lyophilized dosage forms comprising: lyophilized osmotic mediated release barrier-free synthetic nanocarriers comprising an encapsulated osmotically active agent; and lyophilizing agents that provide a vehicle having an osmolality of 200-500 mOsm/kg upon reconstitution of the lyophilized dosage form. The invention further relates to methods comprising: providing osmotic mediated release barrier-free synthetic nanocarriers that comprise an osmotically active agent in an environment having an osmolality ranging from 200-500 mOsm/kg; and administering the osmotic mediated release barrier-free synthetic nanocarriers to a subject.

The invention described herein provides synthetic nanocarriers that do not rely on positive charge to retain osmotically active agents. Such synthetic nanocarriers further provide for rapid release of osmotically active agent(s) from nanocarriers at a relatively high weight percent loading. Mammals, and most other known organisms, maintain a physiologic osmolality around 275-300 mOsm/kg. Slightly hypotonic media and hypertonic media and suspensions of appropriate volume can be administered by most routes, but the range of ~ 200-500 mOsm/kg is preferable as part of the invention to avoid osmolality-driven side effects (e.g., pain, hemolysis). For this reason, in a preferred embodiment, inventive dosage forms are provided that comprise synthetic nanocarriers suspension at near-physiologic osmolality. Once administered (by injection, inhalation, topical application, oral, or other route) the synthetic nanocarriers are preferably deployed into an environment having physiologic-normal osmolality.

Among other aspects, what was surprisingly discovered was the critical role played by balance of osmotic forces in generating and sustaining inventive synthetic nanocarriers comprising osmotically active agents. In embodiments, a steady-state, or near steady-state, condition of the synthetic nanocarriers is preferred during the dosage preparation and for at least part of the period of exposure to the body. Accordingly, the synthetic nanocarriers must be able to sufficiently balance the resulting osmotic pressure gradient across the synthetic nanocarriers without losing essential attributes (e.g., integrity or loading of osmotically active agent(s)). In the presence of an osmotic imbalance, if the synthetic nanocarriers cannot sustain the imbalance and the encapsulated osmotically active agent is at an osmolality greater than the surrounding medium, uncontrolled efflux of the osmotically active agent or loss of nanocarrier structural integrity may occur. Such occurrences result in synthetic nanocarriers having poor performance.

For instance, there is a body of literature regarding the entrapment, encapsulation, and adsorption of nucleic acids in a micro or nanocarrier form. Given the obvious size, water solubility, and net negative charge of nucleic acids, it is unsurprising that the literature largely addresses the use of charge attraction (e.g., cationic chitosan, poly-lysine, or cationic lipids) and diffusive barriers (e.g., intact polymer or lipid walls) to retain oligonucleotide with the carrier. Typical published data is characterized by nanoparticles having 0.1 to 1.0% w/w oligonucleotide loading, a burst release of anywhere from 10 to 80% of the initial load, and then a gradual release of 10-50% of the remaining entrapped oligonucleotide over 5 days to 6 weeks (Malyala et al., 2008; Roman et al., 2008; Diwan et. al 2002; Gvili et al., 2007). These results translate to steady release rates of ~ 0.002 to 1 ug-ON/mg-NC/1-day.

In contrast, there does not appear to be any discussion in the literature of the important role that a balance of osmotic gradients can play in the retention and delivery of nucleic acids or other osmotically active agents in the absence of a cationic or barrier structural component in a synthetic nanocarrier. An advantage of the inventive dosage forms is that it is possible to achieve relatively high loadings of osmotically active agent(s) in the recited synthetic nanocarriers, thus enabling relatively high release rates of osmotically active agent(s) from the synthetic nanocarriers. The ability to provide relatively high release rates of osmotically active agents from synthetic nanocarriers can be important to function. For instance, using model systems, immunization studies demonstrated a correlation between the antibody titers achieved by a CpG-nanocarrier preparation and the rate of CpG release from that nanocarrier in an in vitro test. Synthetic nanocarriers characterized by post-burst release of > 10 µg-CpG/mg-nanocarrier-24 h demonstrated potency in supporting high titers in these studies. It is also observed that increasing the specific release rate, up to at least 30 µg-CpG/mg-nanocarrier-24 h, resulted in increasing antibody titers.

The invention will now be described in more detail below.

### B. Definitions

“Adjuvant” means an agent that does not constitute a specific antigen, but boosts the strength and longevity of an immune response to a concomitantly administered antigen. In embodiments, adjuvants may also be osmotically active agents. Adjuvants may include, but are not limited to, stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (AS04), MPL A of above-mentioned bacteria separately, saponins, such as QS-21,Quil-A, ISCOMs, ISCOMATRIX™, emulsions such as MF59™, Montanide® ISA 51 and ISA 720, AS02 (QS21+squalene+ MPL®), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments.

In embodiments, adjuvants comprise agonists for pattern recognition receptors (PRR), including, but not limited to Toll-Like Receptors (TLRs), specifically TLRs 2, 3, 4, 5, 7, 8, 9 and/or combinations thereof. In other embodiments, adjuvants comprise agonists for Toll-Like Receptors 3, agonists for Toll-Like Receptors 7 and 8, or agonists for Toll-Like Receptor 9; preferably the recited adjuvants comprise imidazoquinolines; such as R848; adenine derivatives, such as those disclosed in U.S. Pat. 6,329,381 (Sumitomo Pharmaceutical Company), U.S. Published Pat. Application 2010/0075995 to Biggadike et al., WO2010/018134, WO 2010/018133, WO 2010/018132, WO 2010/018131, WO 2010/018130 and WO 2008/101867 to Campos et al.; immunostimulatory DNA; or immunostimulatory RNA. In specific embodiments, synthetic nanocarriers incorporate as adjuvants compounds that are agonists for toll-like receptors (TLRs) 7 & 8 (“TLR ⅞ agonists”). Of utility are the TLR ⅞ agonist compounds disclosed in U.S. Pat. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred adjuvants comprise imiquimod and resiquimod (also known as R848). In specific embodiments, an adjuvant may be an agonist for the DC surface molecule CD40. In certain embodiments, to stimulate immunity rather than tolerance, a synthetic nanocarrier incorporates an adjuvant that promotes DC maturation (needed for priming of naive T cells) and the production of cytokines, such as type I interferons, which promote antibody immune responses. In embodiments, adjuvants also may comprise immunostimulatory RNA molecules, such as but not limited to dsRNA, poly I:C or poly I:poly C12U (available as Ampligen ®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an adjuvant may be a TLR-4 agonist, such as bacterial lipopolysacccharide (LPS), VSV-G, and/or HMGB-1. In some embodiments, adjuvants may comprise TLR-5 agonists, such as flagellin, or portions or derivatives thereof, including but not limited to those disclosed in U.S. Pat. 6,130,082, 6,585,980, and 7,192,725. In specific embodiments, synthetic nanocarriers incorporate a ligand for Toll-like receptor (TLR)-9, such as immunostimulatory DNA molecules comprising CpGs, which induce type I interferon secretion, and stimulate T and B cell activation leading to increased antibody production and cytotoxic T cell responses (Krieg et al., CpG motifs in bacterial DNA trigger direct B cell activation. Nature. 1995. 374:546-549; Chu et al. CpG oligodeoxynucleotides act as adjuvants that switch on T helper 1 (Th1) immunity. J. Exp. Med. 1997. 186:1623-1631; Lipford et al. CpG-containing synthetic oligonucleotides promote B and cytotoxic T cell responses to protein antigen: a new class of vaccine adjuvants. Eur. J. Immunol. 1997. 27:2340-2344; Roman et al. Immunostimulatory DNA sequences function as T helper-1-promoting adjuvants.. Nat. Med. 1997. 3:849-854; Davis et al. CpG DNA is a potent enhancer of specific immunity in mice immunized with recombinant hepatitis B surface antigen. J. Immunol. 1998. 160:870-876; Lipford et al., Bacterial DNA as immune cell activator. Trends Microbiol. 1998. 6:496-500; U.S. Pat. 6,207,646 to Krieg et al.; U.S. Pat. 7,223,398 to Tuck et al.; U.S. Pat. 7,250,403 to Van Nest et al.; or U.S. Pat. 7,566,703 to Krieg et al.

In some embodiments, adjuvants may be proinflammatory stimuli released from necrotic cells (e.g., urate crystals). In some embodiments, adjuvants may be activated components of the complement cascade (e.g., CD21, CD35, etc.). In some embodiments, adjuvants may be activated components of immune complexes. The adjuvants also include complement receptor agonists, such as a molecule that binds to CD21 or CD35. In some embodiments, the complement receptor agonist induces endogenous complement opsonization of the synthetic nanocarrier. In some embodiments, adjuvants are cytokines, which are small proteins or biological factors (in the range of 5 kD - 20 kD) that are released by cells and have specific effects on cell-cell interaction, communication and behavior of other cells. In some embodiments, the cytokine receptor agonist is a small molecule, antibody, fusion protein, or aptamer.

“Administering” or “administration” means providing a material to a subject in a manner that is pharmacologically useful.

“Amount effective” is any amount of a composition that produces one or more desired immune responses. This amount can be for in vitro or in vivo purposes. For in vivo purposes, the amount can be one that a health practitioner would believe may have a clinical benefit for a subject in need thereof. In embodiments, therefore, an amount effective is one that a health practitioner would believe may generate an antibody response against any antigen(s) of the inventive compositions provided herein. Effective amounts can be monitored by routine methods. An amount that is effective to produce one or more desired immune responses can also be an amount of a composition provided herein that produces a desired therapeutic endpoint or a desired therapeutic result. Therefore, in other embodiments, the amount effective in one that a clinician would believe would provide a therapeutic benefit (including a prophylactic benefit) to a subject provided herein. Such subjects include those that have or are at risk of having cancer, an infection or infectious disease. Such a subjects include any subject that has or is at risk of having any of the diseases, conditions and/or disorders provide herein.

Amounts effective will depend, of course, on the particular subject being treated; the severity of a condition, disease or disorder; the individual patient parameters including age, physical condition, size and weight; the duration of the treatment; the nature of concurrent therapy (if any); the specific route of administration and like factors within the knowledge and expertise of the health practitioner. These factors are well known to those of ordinary skill in the art and can be addressed with no more than routine experimentation. It is generally preferred that a “maximum dose” be used, that is, the highest safe dose according to sound medical judgment. It will be understood by those of ordinary skill in the art, however, that a patient may insist upon a lower dose or tolerable dose for medical reasons, psychological reasons or for virtually any other reasons. The antigen(s) of any of the inventive compositions provided herein can in embodiments be in an amount effective.

“Antigen” means a B cell antigen or T cell antigen. In embodiments, antigens are coupled to the synthetic nanocarriers. In other embodiments, antigens are not coupled to the synthetic nanocarriers. In embodiments antigens are coadministered with the synthetic nanocarriers. In other embodiments antigens are not coadministered with the synthetic nanocarriers. “Type(s) of antigens” means molecules that share the same, or substantially the same, antigenic characteristics.

“B cell antigen” means any antigen that is or recognized by and triggers an immune response in a B cell (e.g., an antigen that is specifically recognized by a B cell receptor on a B cell). In some embodiments, an antigen that is a T cell antigen is also a B cell antigen. In other embodiments, the T cell antigen is not also a B cell antigen. B cell antigens include, but are not limited to proteins, peptides, small molecules, and carbohydrates. In some embodiments, the B cell antigen comprises a non-protein antigen (i.e., not a protein or peptide antigen). In some embodiments, the B cell antigen comprises a carbohydrate associated with an infectious agent. In some embodiments, the B cell antigen comprises a glycoprotein or glycopeptide associated with an infectious agent. The infectious agent can be a bacterium, virus, fungus, protozoan, or parasite. In some embodiments, the B cell antigen comprises a poorly immunogenic antigen. In some embodiments, the B cell antigen comprises an abused substance or a portion thereof. In some embodiments, the B cell antigen comprises an addictive substance or a portion thereof. Addictive substances include, but are not limited to, nicotine, a narcotic, a cough suppressant, a tranquilizer, and a sedative. In some embodiments, the B cell antigen comprises a toxin, such as a toxin from a chemical weapon or natural sources. The B cell antigen may also comprise a hazardous environmental agent. In some embodiments, the B cell antigen comprises a self antigen. In other embodiments, the B cell antigen comprises an alloantigen, an allergen, a contact sensitizer, a degenerative disease antigen, a hapten, an infectious disease antigen, a cancer antigen, an atopic disease antigen, an autoimmune disease antigen, an addictive substance, a xenoantigen, or a metabolic disease enzyme or enzymatic product thereof.

“Barrier-free” means synthetic nanocarriers that lack a release rate-controlling barrier, located on or within a surface of the synthetic nanocarriers, that controls the release rate of the encapsulated osmotically active agent from the synthetic nanocarriers into an environment surrounding the nanocarriers. In an embodiment, barrier-free synthetic nanocarriers lack a structural element the presence of which would have limited diffusion of osmotically active agents such that an osmotic pressure difference, e.g. allowing the creation of an osmotic pressure difference that would lead to structural disruption of the synthetic nanocarriers, between the interior of the synthetic nanocarriers and the external environment of the synthetic nanocarriers.

“Couple” or “Coupled” or “Couples” (and the like) means to chemically associate one entity (for example a moiety) with another. In some embodiments, the coupling is covalent, meaning that the coupling occurs in the context of the presence of a covalent bond between the two entities. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. In embodiments, encapsulation is a form of coupling.

“Dosage form” means a pharmacologically and/or immunologically active material in a medium, vehicle, carrier, or device suitable for administration to a subject.

“Encapsulate” or “Encapsulated” (and the like) means to couple a first entity or entities to a second entity or entities by completely or partially surrounding some or all of the first entity or entities with the second entity or entities. In embodiments, to encapsulate means to enclose within a synthetic nanocarrier, preferably enclose completely within a synthetic nanocarrier. Most or all of a substance that is encapsulated is not exposed to the local environment external to the synthetic nanocarrier. In other embodiments, no more than 50%, 40%, 30%, 20%, 10% or 5% (weight/weight) is exposed to the local environment. Encapsulation is distinct from absorption, which places most or all of a substance on a surface of a synthetic nanocarrier, and leaves the substance exposed to the local environment external to the synthetic nanocarrier.

“Isolated nucleic acid” means a nucleic acid that may be of varying molecular weight(s) (including oligonucleotides, and polynucleic acids) that is separated from its native environment and present in sufficient quantity to permit its identification or use. An isolated nucleic acid may be one that is (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5‘ and 3‘ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. Any of the nucleic acids provided herein may be isolated.

In embodiments, isolated nucleic acids comprise: immunostimulatory nucleic acids such as immunostimulatory oligonucleotides (including but not limited to both DNA and RNA), small interfering RNA (siRNA), RNA interference (RNAi) oligonucleotides, RNA activating (RNAa) oligonucleotides, micro RNA (miRNA) oligonucleotides, antisense oligonucleotides, aptamers, gene therapy oligonucleotides, plasmids, including their natural and non-natural or modified chemical forms as well as chimeras that include oligonucleotide-based sequences.

While oligonucelotides are macromolecules, their potential to introduce osmolality is significant. A single-strand of an oligonucleotide is a relatively high molecular-weight entity (typically ≥ 2.4 kD at ~ 300 D/nucleotide) with high water solubility (typically ~ 30% w/v). The osmotic contribution of oligonucleotides to a solution is primarily due to counter-ions. The backbone structure of natural nucleic acids, and most unnatural analogs, contributes one negative charge per linkage between base residues, so a nucleotide of “n” monomeric units would have (n-1) associated monovalent counter-ions. For example, a 15 mM solution of a 20-base oligonucleotide with sodium counter-ions has a calculated osmolality of ~300 mOsm/kg. The sodium salt of an oligonucleotide near its solubility limit in water may contribute around 1000 mOsm/kg.

In a preferred embodiment, isolated nucleic acids may comprise immunostimulatory oligonucleotides(s) such as immunostimulatory DNA oligonucleotides comprising 5‘ - CG - 3“ motifs or immunostimulatory RNA oligonucleotides. In an embodiment, any cytosine nucleotides (“C″) present in a 5‘ - CG - 3” motif in immunostimulatory oligonucleotides are unmethylated. C present in parts of the immunostimulatory oligonucleotides other than in 5‘ - CG - 3“ motifs may be methylated, or may be unmethylated. In embodiments, the recited immunostimulatory oligonucleotides possess a phosphodiester backbone that is not modified to incorporate phosphorothioate bonds, preferably the phosphodiester backbone is free of phosphorothioate bonds. In other embodiments, the immunostimulatory oligonucleotides‘ phosphodiester backbone comprises no stabilizing chemical modifications that function to stabilize the phosphodiester backbone under physiological conditions.

“Isolated peptide” means a peptide that may be of varying molecular weight(s) (including peptides, oligopeptides, polypeptides, and proteins) that is separated from its native environment and present in sufficient quantity to permit its identification or use. This means, for example, the peptide may be (i) selectively produced by expression cloning or (ii) purified as by chromatography or electrophoresis. Isolated peptides may be, but need not be, substantially pure. Because an isolated peptide may be admixed with a pharmaceutically acceptable carrier in a pharmaceutical preparation, the peptide may comprise only a small percentage by weight of the preparation. The peptide is nonetheless isolated in that it has been separated from the substances with which it may be associated in living systems, i.e., isolated from other peptides. Any of the peptides provided herein may be isolated. In embodiments, isolated peptides comprise osmotically active: immunomodulatory peptides such as MHC Class I or MHC Class II binding peptides, antigenic peptides, hormones and hormone mimetics, ligands, antibacterial and antimicrobial peptides, anti-coagulation peptides, and enzyme inhibitors.

“Isolated saccharide” means a saccharide that may be of varying molecular weight(s) (including monosaccharides, disaccharides, trisaccharides, oligosaccharides, polysaccharides, and the like) that is separated from its native environment and present in sufficient quantity to permit its identification or use. This means, for example, the saccharide may be (i) selectively produced by synthetic methods or (ii) purified as by chromatography or electrophoresis. Isolated saccharides may be, but need not be, substantially pure. Because an isolated saccharide may be admixed with a pharmaceutically acceptable carrier in a pharmaceutical preparation, the saccharide may comprise only a small percentage by weight of the preparation. The saccharide is nonetheless isolated in that it has been separated from the substances with which it may be associated in living systems, i.e., isolated from other saccharides or peptides. Any of the saccharides provided herein may be isolated. In embodiments, isolated saccharides comprise osmotically active: antigenic saccharides (e.g., saccharides characteristic of a pathogenic or xenobiotic organism), lipopolysaccharides, protein or peptide mimetic saccharides, cell surface targeting saccharides, anticoagulants, anti-inflammatory saccharides, anti-proliferative saccharides, including their natural and modified forms.

“Lyophilized dosage form” means a dosage form that has undergone lyophilization.

“Lyophilized osmotic mediated release barrier-free synthetic nanocarriers” means osmotic mediated release barrier-free synthetic nanocarriers that have undergone lyophilization.

“Lyophilizing agents” mean substances that are added to a dosage form to facilitate lyophilization of the dosage form, or reconstitution of the dosage form once lyophilized. In embodiments, lyophilizing agents may also be osmotically active agents, and may be selected so as to provide a vehicle having an osmolality of 200-500 mOsm/kg upon reconstitution of the lyophilized dosage form. In embodiments, lyophilizing agents comprise salts and buffering agents (such as NaCl, NaPO₄, or Tris), simple or complex carbohydrates (such as sucrose, dextrose, dextran, or carboxymethyl cellulose), polyols (such as mannitol, sorbitol, glycerol, polyvinyl alcohol), pH adjustment agents (such as HCl, NaOH, or sodium citrate), chelating and antioxidant agents (such as EDTA, ascorbic acid, alpha-tocopherol), stabilizers and preservatives (such as gelatin, glycine, histidine, or benzyl alcohol), surfactants (such as polysorbate 80, sodium deoxycholate, or Triton X-100.

“Maximum dimension of a synthetic nanocarrier” means the largest dimension of a nanocarrier measured along any axis of the synthetic nanocarrier. “Minimum dimension of a synthetic nanocarrier” means the smallest dimension of a synthetic nanocarrier measured along any axis of the synthetic nanocarrier. For example, for a spheroidal synthetic nanocarrier, the maximum and minimum dimension of a synthetic nanocarrier would be substantially identical, and would be the size of its diameter. Similarly, for a cuboidal synthetic nanocarrier, the minimum dimension of a synthetic nanocarrier would be the smallest of its height, width or length, while the maximum dimension of a synthetic nanocarrier would be the largest of its height, width or length. In an embodiment, a minimum dimension of at least 75%, preferably at least 80%, more preferably at least 90%, of the synthetic nanocarriers in a sample, based on the total number of synthetic nanocarriers in the sample, is greater than 100 nm. In an embodiment, a maximum dimension of at least 75%, preferably at least 80%, more preferably at least 90%, of the synthetic nanocarriers in a sample, based on the total number of synthetic nanocarriers in the sample, is equal to or less than 5 µm. Preferably, a minimum dimension of at least 75%, preferably at least 80%, more preferably at least 90%, of the synthetic nanocarriers in a sample, based on the total number of synthetic nanocarriers in the sample, is greater than 110 nm, more preferably greater than 120 nm, more preferably greater than 130 nm, and more preferably still greater than 150 nm. Aspects ratios of the maximum and minimum dimensions of inventive synthetic nanocarriers may vary depending on the embodiment. For instance, aspect ratios of the maximum to minimum dimensions of the synthetic nanocarriers may vary from 1:1 to 1,000,000:1, preferably from 1:1 to 100,000:1, more preferably from 1:1 to 1000:1, still preferably from 1:1 to 100:1, and yet more preferably from 1:1 to 10:1. Preferably, a maximum dimension of at least 75%, preferably at least 80%, more preferably at least 90%, of the synthetic nanocarriers in a sample, based on the total number of synthetic nanocarriers in the sample is equal to or less than 3 µm, more preferably equal to or less than 2 µm, more preferably equal to or less than 1 µm, more preferably equal to or less than 800 nm, more preferably equal to or less than 600 nm, and more preferably still equal to or less than 500 nm. In preferred embodiments, a minimum dimension of at least 75%, preferably at least 80%, more preferably at least 90%, of the synthetic nanocarriers in a sample, based on the total number of synthetic nanocarriers in the sample, is equal to or greater than 100 nm, more preferably equal to or greater than 120 nm, more preferably equal to or greater than 130 nm, more preferably equal to or greater than 140 nm, and more preferably still equal to or greater than 150 nm. Measurement of synthetic nanocarrier sizes is obtained by suspending the synthetic nanocarriers in a liquid (usually aqueous) media and using dynamic light scattering (DLS) (e.g. using a Brookhaven ZetaPALS instrument). For example, a suspension of synthetic nanocarriers can be diluted from an aqueous buffer into purified water to achieve a final synthetic nanocarrier suspension concentration of approximately 0.01 to 0.1 mg/mL. The diluted suspension may be prepared directly inside, or transferred to, a suitable cuvette for DLS analysis. The cuvette may then be placed in the DLS, allowed to equilibrate to the controlled temperature, and then scanned for sufficient time to aquire a stable and reproducible distribution based on appropriate inputs for viscosity of the medium and refractive indicies of the sample. The effective diameter, or mean of the distribution, is then reported.

“Osmotic mediated release” means release of osmotically active agent(s) from synthetic nanocarriers in a manner that satisfies the following in vitro test:

Reconstitute or dilute the dosage form to be tested into near neutral-pH aqueous media (e.g., pH 7.4) at 25° C. yielding a composition with osmolality between 270-330 mOsm/kg, referred to as the Near-Physiologic Osmolality Media. Then, dilute a sample of the Near-Physiologic Osmolality Media by 9x in either purified water or phosphate buffered saline media (e.g., to a final osmolality of approximately 25-35 mOsm/kg) to yield the Low-Osmolality Media. Next, measure concentration of osmotically active agent in the Near-Physiologic Osmolality Media (e.g., by OD₂₆₀ for nucleic acids) and then after 2 hours of gentle agitation at 25° C. in the Low-Osmolality Media. If the release (e.g., the total osmotically active agent released into solution over 2 hours) is significantly greater in the Low-Osmolality Media than in the Near-Physiologic Osmolality Media (preferably Release_{Low-Osmolality} _{Media} > 1.5 x Release_{Near-Physiologic} _{Osmolality} _{Media}, more preferably Release_{Low-Osmoiality} _{Media} > 5 x Release_{Near-Physiologic} _{Osmolality} _{Media}), even more preferably Release_{Low-Osmolality} _{Media} > 10 x Release_{Near-Physiologic} _{Osmolality} _{Media}) then the test is positive for an osmotic mediated release.

“Osmotically active agent” means a substance having solubility in an aqueous solvent. The osmotically active agent(s) may be present in the synthetic nanocarriers in varying amounts. In embodiments, the osmotically active agent is present in the synthetic nanocarriers in an amount of about 2, or 3, or 4, or 5, or 6, or 7, or 8 weight percent, based on the total theoretical weight of the synthetic nanocarriers. The osmotically active agent may comprise more than one molecular entity, including specifically associated soluble materials such as counter-ions. In embodiments, the osmotically active agent comprises an isolated nucleic acid, a polymer, an isolated peptide, an isolated saccharide, macrocycle, or ions, cofactors, coenzymes, ligands, hydrophobically-paired agents, or hydrogen-bond donors or acceptors of any of the above, that are specifically, but non-covalently, associated with any of the foregoing. Osmotically active agents may have a variety of functions in the inventive synthetic nanocarriers. Accordingly osmotically active agents may comprise antigens, adjuvants, or substances with other immunostimulatory or immunomodulatory functions. The osmotic contribution of a osmotically-active agent to an aqueous solution can be measured by any of several accepted technologies, not limited to but including, vapor pressure depression, freezing point depression, or membrane osmometers. Specific types of osmometers conventionally available include the Wescor Vapro II vapor pressure osmometer model series, Advanced Instruments 3250 freezing point osmometer model series, and the UIC model 231 membrane osmometer.

“pH triggered osmotic mediated release barrier-free synthetic nanocarriers” means osmotic mediated release barrier-free synthetic nanocarriers that release significantly greater amounts of the osmotically-active agent within 1 hour of introduction into an isotonic medium of pH 4.5, or of pH 10.5, than is released into an isotonic medium of pH 7.4. The release is said to be pH triggered if it satisfies the following in vitro test:

Reconstitute or dilute the dosage form to be tested into near neutral-pH aqueous media (e.g., pH 7.4) at 25° C. yielding a composition with osmolality between 270-330 mOsm/kg, referred to as the Near-Physiologic Osmolality and Near-Neutral pH Media and measure concentration of the osmotically-active agent upon dilution and after 2 hours of gentle agitation at 37° C. Calculate the total amount of osmotically active agent released over 2 hours, and define the net amount released per 2 hours as the Near-Physiologic Osmolality and Near-Neutral pH Release Rate. Next, repeat the same process in pH 4.5 (or into pH 10.5) aqueous media with osmolality between 270-330 mOsm/kg, referred to as the Acidic (or Basic) Near-Physiologic Osmolality Media. Calculate the total amount of osmotically active agent released over 2 hours, and define the net amount released per 2 hours as the Acidic (or Basic) Near-Physiologic Osmolality Release Rate. If the release rate (e.g., the total osmotically active agent released into solution over 2 hours) is significantly greater in the Acidic (or Basic) Near-Physiologic Osmolality Media than in the Near-Physiologic Osmolality and Near-Neutral pH Media (preferably Release_{Acidic} ₍ₒᵣ _{Basic)} _{Media} > 1.2 x Release_{Near-Neutral}_{Media}, more preferably Release _{Acidic} ₍ₒᵣ _{Basic)} _{Media} > 1.5 x Release_{Near-Neutral}_{Media}, even more preferably Release _{Acidic} ₍ₒᵣ _{Basic)} _{Media} > 3x Release_{Near-Neutral Media}) then the test is positive for a pH triggered osmotic mediated release.

“Pharmaceutically acceptable excipient” means a pharmacologically inactive material used together with the recited synthetic nanocarriers to formulate the inventive compositions. Pharmaceutically acceptable excipients comprise a variety of materials known in the art, including but not limited to saccharides (such as glucose, lactose, and the like), preservatives such as antimicrobial agents, reconstitution aids, colorants, saline (such as phosphate buffered saline), and buffers.

“Polymer” means a synthetic compound comprising large molecules made up of a covalently linked series of repeated simple (co)monomers. In embodiments, polymer comprises osmotically active: dendrimers, polylactic acids, polyglycolic acids, poly lactic-co-glycolic acids, polycaprolactams, polyethylene glycols, polyacrylates, polymethacrylates, and co-polymers and/or combinations of any of the above.

“Release” or “Release Rate” means the rate that an entrapped substance transfers from a synthetic nanocarrier into local environment, such as a surrounding release media. First, the synthetic nanocarrier is prepared for the release testing by placing into the appropriate release media. This is generally done by exchanging a buffer after centrifugation to pellet the synthetic nanocarrier and reconstitution of the synthetic nanocarriers under a mild condition. The assay is started by placing the sample at 37° C. in an appropriate temperature-controlled apparatus. A sample is removed at various time points.

The synthetic nanocarriers are separated from the release media by centrifugation to pellet the synthetic nanocarriers. The release media is assayed for the substance that has been released from the synthetic nanocarriers. The substance is measured using HPLC to determine the content and quality of the substance. The pellet containing the remaining entrapped substance is dissolved in solvents or hydrolyzed by base to free the entrapped substance from the synthetic nanocarriers. The pellet-contained substance is then also measured by HPLC after dissolution or destruction of the pellet to determine the content and quality of the substance that has not been released at a given time point.

The mass balance is closed between substance that has been released into the release media and what remains in the synthetic nanocarriers. Data are presented as the fraction released or as the net release presented as micrograms released over time.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Albumin nanoparticles are generally included as synthetic nanocarriers, however in certain embodiments the synthetic nanocarriers do not comprise albumin nanoparticles. In embodiments, inventive synthetic nanocarriers do not comprise chitosan.

A synthetic nanocarrier can be, but is not limited to, one or a plurality of lipid-based nanoparticles, polymeric nanoparticles, dendrimers, virus-like particles, peptide or protein-based particles (such as albumin nanoparticles), ceramic-based nanoparticles (e.g. semi-porous silicon nanoparticles), hydrogel nanoparticles, polysaccharide-based nanoparticles, and/or nanoparticles that are developed using a combination of nanomaterials such as lipid-polymer nanoparticles. Synthetic nanocarriers may be a variety of different shapes, including but not limited to spheroidal, cuboidal, pyramidal, oblong, cylindrical, toroidal, and the like. Synthetic nanocarriers according to the invention comprise one or more surfaces. Exemplary synthetic nanocarriers that can be adapted for use in the practice of the present invention comprise: (1) the biodegradable nanoparticles disclosed in U.S. Pat. 5,543,158 to Gref et al., (2) the polymeric nanoparticles of Published U.S. Pat. Application 20060002852 to Saltzman et al., (3) the lithographically constructed nanoparticles of Published U.S. Pat. Application 20090028910 to DeSimone et al., (4) the disclosure of WO 2009/051837 to von Andrian et al., (5) the protein nanoparticles disclosed in Published U.S. Pat. Application 20090226525 to de los Rios et al., (6) the virus-like particles disclosed in published U.S. Pat. Application 20060222652 to Sebbel et al., (7) the nucleic acid coupled virus-like particles disclosed in published U.S. Pat. Application 20060251677 to Bachmann et al., (8) the virus-like particles disclosed in WO2010047839A1 or WO2009106999A2, or (9) the nanoprecipitated nanoparticles disclosed in P. Paolicelli et al., “Surface-modified PLGA-based Nanoparticles that can Efficiently Associate and Deliver Virus-like Particles” Nanomedicine. 5(6):843-853 (2010). In embodiments, synthetic nanocarriers may possess an aspect ratio greater than 1:1, 1:1.2, 1:1.5, 1:2, 1:3, 1:5, 1:7, or greater than 1:10.

Synthetic nanocarriers according to the invention that have a minimum dimension of equal to or less than about 100 nm, preferably equal to or less than 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, preferably equal to or less than 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than 100 nm, do not comprise a surface that activates complement or alternatively comprise a surface that consists essentially of moieties that do not activate complement. In embodiments, synthetic nanocarriers exclude virus-like particles. In embodiments, when synthetic nanocarriers comprise virus-like particles, the virus-like particles comprise non-natural adjuvant (meaning that the VLPs comprise an adjuvant other than naturally occurring RNA generated during the production of the VLPs).

“T cell antigen” means any antigen that is recognized by and triggers an immune response in a T cell (e.g., an antigen that is specifically recognized by a T cell receptor on a T cell or an NKT cell via presentation of the antigen or portion thereof bound to a Class I or Class II major histocompatability complex molecule (MHC), or bound to a CD1 complex). In some embodiments, an antigen that is a T cell antigen is also a B cell antigen. In other embodiments, the T cell antigen is not also a B cell antigen. T cell antigens generally are proteins or peptides. T cell antigens may be an antigen that stimulates a CD8+ T cell response, a CD4+ T cell response, or both. The nanocarriers, therefore, in some embodiments can effectively stimulate both types of responses.

In some embodiments the T cell antigen is a T helper cell antigen (i.e. one that can generate an enhanced response to a B cell antigen, preferably an unrelated B cell antigen, through stimulation of T cell help). In embodiments, a T helper cell antigen may comprise one or more peptides obtained or derived from tetanus toxoid, Epstein-Barr virus, influenza virus, respiratory syncytial virus, measles virus, mumps virus, rubella virus, cytomegalovirus, adenovirus, diphtheria toxoid, or a PADRE peptide (known from the work of Sette et al. U.S. Pat. 7,202,351). In other embodiments, a T helper cell antigen may comprise one or more lipids, or glycolipids, including but not limited to: α-galactosylceramide (α-GalCer), α-linked glycosphingolipids (from Sphingomonas spp.), galactosyl diacylglycerols (from Borrelia burgdorferi), lypophosphoglycan (from Leishmania donovani), and phosphatidylinositol tetramannoside (PIM4) (from Mycobacterium leprae). For additional lipids and/or glycolipids useful as a T helper cell antigen, see V. Cerundolo et al., “Harnessing invariant NKT cells in vaccination strategies.” Nature Rev Immun, 9:28-38 (2009). In embodiments, CD4+ T-cell antigens may be derivatives of a CD4+ T-cell antigen that is obtained from a source, such as a natural source. In such embodiments, CD4+ T-cell antigen sequences, such as those peptides that bind to MHC II, may have at least 70%, 80%, 90%, or 95% identity to the antigen obtained from the source. In embodiments, the T cell antigen, preferably a T helper cell antigen, may be coupled to, or uncoupled from, a synthetic nanocarrier.

“Vaccine” means a composition of matter that improves the immune response to a particular pathogen or disease. A vaccine typically contains factors (such as antigens, adjuvants, and the like) that stimulate a subject’s immune system to recognize a specific antigen as foreign and eliminate it from the subject’s body. A vaccine also establishes an immunologic ‘memory’ so the antigen will be quickly recognized and responded to if a person is re-challenged. Vaccines can be prophylactic (for example to prevent future infection by any pathogen), or therapeutic (for example a vaccine against a tumor specific antigen for the treatment of cancer). In embodiments, a vaccine may comprise dosage forms according to the invention.

“Vehicle” means a material of little or no therapeutic value used to convey synthetic nanocarriers for administration. In a preferred embodiment, vehicles according to the invention comprise those vehicles having an osmolality of 200-500 mOsm/kg.

### C. Inventive Compositions

A wide variety of synthetic nanocarriers can be used according to the invention. In some embodiments, synthetic nanocarriers are spheres or spheroids. In some embodiments, synthetic nanocarriers are flat or plate-shaped. In some embodiments, synthetic nanocarriers are cubes or cubic. In some embodiments, synthetic nanocarriers are ovals or ellipses. In some embodiments, synthetic nanocarriers are cylinders, cones, or pyramids.

In some embodiments, it is desirable to use a population of synthetic nanocarriers that is relatively uniform in terms of size, shape, and/or composition so that each synthetic nanocarrier has similar properties. For example, at least 80%, at least 90%, or at least 95% of the synthetic nanocarriers, based on the total number of synthetic nanocarriers, may have a minimum dimension or maximum dimension that falls within 5%, 10%, or 20% of the average diameter or average dimension of the synthetic nanocarriers. In some embodiments, a population of synthetic nanocarriers may be heterogeneous with respect to size, shape, and/or composition.

Synthetic nanocarriers can be solid or hollow and can comprise one or more layers -so long as the layers do not act as a release rate-controlling barrier, located on or within a surface of the synthetic nanocarriers, that controls the release rate of the encapsulated osmotically active agent from the synthetic nanocarriers into an environment surrounding the nanocarriers. In some embodiments, each layer has a unique composition and unique properties relative to the other layer(s). To give but one example, synthetic nanocarriers may have a core/shell structure, wherein the core is one layer (e.g. a polymeric core) and the shell is a second layer (e.g. a lipid bilayer or monolayer). Synthetic nanocarriers may comprise a plurality of different layers.

In some embodiments, synthetic nanocarriers may optionally comprise one or more lipids, so long as the lipids do not function as a release rate-controlling barrier, located on or within a surface of the synthetic nanocarriers, that controls the release rate of the encapsulated osmotically active agent from the synthetic nanocarriers into an environment surrounding the nanocarriers. In some embodiments, a synthetic nanocarrier may comprise a liposome. In some embodiments, a synthetic nanocarrier may comprise a lipid bilayer. In some embodiments, a synthetic nanocarrier may comprise a lipid monolayer. In some embodiments, a synthetic nanocarrier may comprise a micelle. In some embodiments, a synthetic nanocarrier may comprise a core comprising a polymeric matrix surrounded by a lipid layer (e.g., lipid bilayer, lipid monolayer, etc.). In some embodiments, a synthetic nanocarrier may comprise a non-polymeric core (e.g., viral particle, proteins, nucleic acids, carbohydrates, etc.) surrounded by a lipid layer (e.g., lipid bilayer, lipid monolayer, etc.).

In some embodiments, synthetic nanocarriers can comprise one or more polymers. In some embodiments, such a polymer can be surrounded by a coating layer (e.g., liposome, lipid monolayer, micelle, etc.) so long as the coating layer does not function as a release rate-controlling barrier, located on or within a surface of the synthetic nanocarriers, that controls the release rate of the encapsulated osmotically active agent from the synthetic nanocarriers into an environment surrounding the nanocarriers. In some embodiments, various elements of the synthetic nanocarriers can be coupled with the polymer.

In some embodiments, an element, such as an immunofeature surface, targeting moiety, and/or oligonucleotide can be covalently associated with a polymeric matrix. In some embodiments, covalent association is mediated by a linker. In some embodiments, an element, such as an immunofeature surface, targeting moiety, and/or oligonucleotide can be noncovalently associated with a polymeric matrix. For example, in some embodiments, element, such as an immunofeature surface, targeting moiety, and/or oligonucleotide can be encapsulated within, surrounded by, and/or dispersed throughout a polymeric matrix. Alternatively or additionally, an element, such as an immunofeature surface, targeting moiety, and/or nucleotide can be associated with a polymeric matrix by hydrophobic interactions, charge interactions, van der Waals forces, etc.

A wide variety of polymers and methods for forming polymeric matrices therefrom are known conventionally. In general, a polymeric matrix comprises one or more polymers. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with the present invention are organic polymers.

Examples of polymers suitable for use in the present invention include, but are not limited to polyethylenes, polycarbonates (e.g. poly(1,3-dioxan-2one)), polyanhydrides (e.g. poly(sebacic anhydride)), polypropylfumerates, polyamides (e.g. polycaprolactam), polyacetals, polyethers, polyesters (e.g., polylactide, polyglycolide, polylactide-co-glycolide, polycaprolactone, polyhydroxyacid (e.g. poly(β-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. coupled) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5543158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanocarriers may optionally comprise one or more amphiphilic entities. In some embodiments, an amphiphilic entity can promote the production of synthetic nanocarriers with increased stability, improved uniformity, or increased viscosity. Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol;sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used in the production of synthetic nanocarriers to be used in accordance with the present invention.

In some embodiments, synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the inventive synthetic nanocarriers do not comprise (or specifically exclude) carbohydrates, such as a polysaccharide. In certain embodiments, the carbohydrate may comprise a carbohydrate derivative such as a sugar alcohol, including but not limited to mannitol, sorbitol, xylitol, erythritol, maltitol, and lactitol.

Compositions according to the invention comprise inventive synthetic nanocarriers in combination with pharmaceutically acceptable excipients, such as preservatives, buffers, saline, or phosphate buffered saline. The compositions may be made using conventional pharmaceutical manufacturing and compounding techniques to arrive at useful dosage forms. In an embodiment, inventive synthetic nanocarriers are suspended in sterile saline solution for injection together with a preservative.

In embodiments, when preparing synthetic nanocarriers as carriers for antigens and/or adjuvants for use in vaccines, methods for coupling the antigens and/or adjuvants to the synthetic nanocarriers may be useful. If the adjuvant is a small molecule it may be of advantage to attach the antigens and/or adjuvants to polymers prior to the assembly of the synthetic nanocarriers. In embodiments, it may also be an advantage to prepare the synthetic nanocarriers with surface groups that are used to couple the antigens and/or adjuvants to the synthetic nanocarriers through the use of these surface groups rather than attaching the antigens and/or adjuvants to polymers and then using the polymer conjugates in the construction of synthetic nanocarriers.

In certain embodiments, the coupling can be a covalent linker. In embodiments, antigens and/or adjuvants can be covalently coupled to an external synthetic nanocarrier surface via a 1,2,3-triazole linker formed by the 1,3-dipolar cycloaddition reaction of azido groups on the surface of the nanocarrier with antigen and/or adjuvant containing an alkyne group or by the 1,3-dipolar cycloaddition reaction of alkynes on the surface of the nanocarrier with antigens or adjuvants containing an azido group. Such cycloaddition reactions are preferably performed in the presence of a Cu(I) catalyst along with a suitable Cu(I)-ligand and a reducing agent to reduce Cu(II) compound to catalytic active Cu(I) compound. This Cu(I)-catalyzed azide-alkyne cycloaddition (CuAAC) can also be referred as the click reaction. Additionally, the covalent coupling may comprise a covalent linker that comprises an amide linker, a disulfide linker, a thioether linker, a hydrazone linker, a hydrazide linker, an imine or oxime linker, an urea or thiourea linker, an amidine linker, an amine linker, and a sulfonamide linker.

Elements of the inventive synthetic nanocarriers (such as moieties of which an immunofeature surface is comprised, targeting moieties, polymeric matrices, antigens and the like) may be coupled to the overall synthetic nanocarrier, e.g., by one or more covalent bonds, or may be coupled by means of one or more linkers. Additional methods of functionalizing synthetic nanocarriers may be adapted from Published U.S. Pat. Application 2006/0002852 to Saltzman et al., Published U.S. Pat. Application 2009/0028910 to DeSimone et al., or Published International Patent Application WO/2008/127532 A1 to Murthy et al.

Alternatively or additionally, synthetic nanocarriers can be coupled to immunofeature surfaces, targeting moieties, adjuvants, various antigens, and/or other elements directly or indirectly via non-covalent interactions. In non-covalent embodiments, the non-covalent coupling is mediated by non-covalent interactions including but not limited to charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof. Such couplings may be arranged to be on an external surface or an internal surface of an inventive synthetic nanocarrier. In embodiments, encapsulation and/or absorption is a form of coupling.

For detailed descriptions of additional available conjugation methods, see Hermanson G T “Bioconjugate Techniques”, 2nd Edition Published by Academic Press, Inc., 2008.

### D. Methods of Making and Using the Inventive Compositions and Related Methods

In an embodiment, a novel factor in creating and maintaining the inventive synthetic nanocarriers is the use of osmotic balancing at near-physiologic osmolality during processing and storage. In an embodiment, during the assembly and dosage preparation of inventive synthetic nanocarriers that comprise osmotically active agent(s), osmolality plays an important role.

Balance of the osmolality (e.g., between inner and outer aqueous phases) can be important for efficient loading during preparation of inventive synthetic nanocarrier formulations. The inventors have recognized that optimal efficacy of an inventive nanocarrier preparation as a means to administer osmotically active agents to a biological system implies an optimum preparative osmolality corresponding approximately to that of the physiologic target. In an embodiment, maintaining osmotic balance at a near-physiological level throughout processing and formulation provides for optimized inventive synthetic nanocarriers with respect to encapsulation efficiency, loading stability during storage and dosing, and effective delivery.

In embodiments according to the invention, osmotic mediated release barrier-free nanocarriers are formed in environments having an osmolality ranging from 200-500 mOsm/kg. Environments having an osmolality in this range mimic the local osmotic environment found in subjects to whom the inventive dosage forms might be administered.

Environments according to the invention may be prepared at a specified osmolality using a variety of techniques. For instance, the concentration of ions having osmotic activity may be titrated up or down to achieve the desired osmolality. Materials that can be used to increase or decrease environmental osmolality comprise salts and buffering agents (such as NaCl, CaCl₂, or NaPO₄), simple or complex carbohydrates (such as sucrose, dextrose, dextran, or sodium carboxymethyl cellulose), polyols (such as sorbitol, glycerol, or polyvinyl alcohol), pH adjustment agents (such as HCl, NaOH, or acetic acid), amino acids and peptides (such as glycine, histidine, and ), chelating or antioxidant agents (such as EDTA, ascorbic acid), vitamins, dissolved gasses, water-soluble polymers (e.g., polyvinylpyrrolidone, poloxamer, or polyethyleneglycol), and preservative and antimicrobials (such as benzoic acid). The agents that contribute to the osmolality of processing media or environments may have additional functional roles in addition to osmolality adjustment. To reduce osmolality, dilution is the traditional method, for example diluting an environment with water or with another aqueous medium having lower osmolality. Furthermore, lower osmolality could be induced in the environment of the nanocarrier (or its in-process form) by removing osmotic agents from the nanocarrier media, for example by precipitation or by liquid-liquid extraction. For example, in an embodiment, a condensing agent such as chitosan could be added to the aqueous media which may cause soluble ions to precipitate. Chelating agents and resins may also be introduced into the environment to reduce the net solute concentration. An example of liquid-liquid extraction would include the contact of an organic phase (such as dichloromethane) with the aqueous environment such that a water-soluble agent will partition, at least in part, into the dichloromethane phase (e.g., benzoic acid). The osmolality of an aqueous solution can be measured by any of several accepted technologies, not limited to but including, vapor pressure depression, freezing point depression, or membrane osmometers. As noted elsewhere herein, useful types of osmometers include the Wescor Vapro II vapor pressure osmometer model series, Advanced Instruments 3250 freezing point osmometer model series, and the UIC model 231 membrane osmometer.

In embodiments, once the osmotic mediated release barrier-free synthetic nanocarriers are formed, they may be maintained in an environment that has an osmolality ranging from 200-500 mOsm/kg. This may help to preserve the integrity of the synthetic nanocarriers, and also reduce or prevent undesirable or premature release of the osmotically active agent during manufacture of the osmotic mediated release barrier-free synthetic nanocarriers. In embodiments, the specific environment may be changed, using methods like dialysis or centrifugation followed by resuspension. In other embodiments, the environment in which the osmotic mediated release barrier-free synthetic nanocarriers are formed, and the environment in which the osmotic mediated release barrier-free synthetic nanocarriers are maintained, are the same. Situations in which the environment is changed or kept the same may be driven by the nature of the manufacturing processes involved, the type of synthetic nanocarriers being manufactured, and the nature of the osmotically active agent(s), among other factors. The osmolality of the environment can be monitored using various measurement techniques as described elsewhere herein, and osmolality can be maintained using titration of various reagents again as described elsewhere herein.

In embodiments, the formed osmotic mediated release barrier-free synthetic nanocarriers may be processed in an environment having an osmolality ranging from 200-500 mOsm/kg. In embodiments, processing can comprise a number of different unit operations that may comprise: washing the synthetic nanocarriers, centrifuging the synthetic nanocarriers, filtering the synthetic nanocarriers, concentrating or diluting the synthetic nanocarriers, freezing the synthetic nanocarriers, drying the synthetic nanocarriers, combining the synthetic nanocarriers with other synthetic nanocarriers or with additive agents or excipients, adjusting the pH or buffer environment of the synthetic nanocarriers, entrapping the synthetic nanocarriers in a gel or high-viscosity medium, resuspending the synthetic nanocarriers, surface modifying the synthetic nanocarriers covalently or by physical processes such as coating or annealing, impregnating or doping the synthetic nanocarriers with active agents or excipients, sterilizing the synthetic nanocarriers, reconstituting the synthetic nanocarriers for administration, or combinations of any of the above. Additionally, in embodiments the formed osmotic mediated release barrier-free synthetic nanocarriers may be stored in an environment having an osmolality ranging from 200-500 mOsm/kg. Again, processing in such an environment may help to preserve the integrity of the synthetic nanocarriers, and also reduce or prevent undesirable or premature release of the osmotically active agent during manufacture of the osmotic mediated release barrier-free synthetic nanocarriers. The specific materials making up the processing or storage environments may be changed or kept the same, so long as the environment is maintained at an osmolality ranging from 200-500 mOsm/kg.

In embodiments, the formed osmotic mediated release barrier-free synthetic nanocarriers may be formulated into a dosage form that maintains the formed osmotic mediated release barrier-free synthetic nanocarriers in an environment having an osmolality ranging from 200-500 mOsm/kg. In embodiments, the environment may comprise a vehicle that is formulated to have osmolality ranging from 200-500 mOsm/kg. The vehicle’s molality may be established using techniques and materials disclosed elsewhere herein for creating and/or maintaining an environmental osmolality, with the exception that the materials and techniques chosen must be suitable for the type of dosage form in question. For instance, materials used to increase the osmolality of the vehicle in an injectable dosage form should be suitable for use in parenteral dosage forms. Suspension, gel, or frozen suspension dosage forms may be prepared to an appropriate osmolality with the inclusion of osmolality adjustment agents. Examples of these include, but are not limited to, water-soluble buffers, salts, carbohydrates, polyols, amino acids, ions, and co-solvents that contribute to the osmotic pressure of the dosage form, along with other such agents noted elsewhere herein. If the dosage form is to be lyophilized, conventional lyophilization equipment run at conventional settings can be used in the practice of the present invention.

In embodiments, dosage forms that are to be administered to subjects comprise osmotic mediated release barrier-free synthetic nanocarriers that are processed only in environments having an osmolality ranging from 200-500 mOsm/kg, thus preventing undesirable release (e.g. premature or in an inappropriate environment) of osmotically active agent. Such processing comprises: washing the synthetic nanocarriers, centrifuging the synthetic nanocarriers, filtering the synthetic nanocarriers, concentrating or diluting the synthetic nanocarriers, freezing the synthetic nanocarriers, drying the synthetic nanocarriers, combining the synthetic nanocarriers with other synthetic nanocarriers or with additive agents or excipients, adjusting the pH or buffer environment of the synthetic nanocarriers, entrapping the synthetic nanocarriers in a gel or high-viscosity medium, resuspending the synthetic nanocarriers, surface modifying the synthetic nanocarriers covalently or by physical processes such as coating or annealing, impregnating or doping the synthetic nanocarriers with active agents or excipients, sterilizing the synthetic nanocarriers, reconstituting the synthetic nanocarriers for administration, or combinations of any of the above.

Synthetic nanocarriers may be prepared using a wide variety of methods known in the art. For example, synthetic nanocarriers can be formed by methods as nanoprecipitation, flow focusing using fluidic channels, spray drying, single and double emulsion solvent evaporation, solvent extraction, phase separation, milling, microemulsion procedures, microfabrication, nanofabrication, sacrificial layers, simple and complex coacervation, and other methods well known to those of ordinary skill in the art. Alternatively or additionally, aqueous and organic solvent syntheses for monodisperse semiconductor, conductive, magnetic, organic, and other nanomaterials have been described (Pellegrino et al., 2005, Small, 1:48; Murray et al., 2000, Ann. Rev. Mat. Sci., 30:545; and Trindade et al., 2001, Chem. Mat., 13:3843). Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5578325 and 6007845; P. Paolicelli et al., “Surface-modified PLGA-based Nanoparticles that can Efficiently Associate and Deliver Virus-like Particles” Nanomedicine. 5(6):843-853 (2010)).

Various materials may be encapsulated into synthetic nanocarriers as desirable using a variety of methods including but not limited to C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8- 21 (2006); P. Paolicelli et al., “Surface-modified PLGA-based Nanoparticles that can Efficiently Associate and Deliver Virus-like Particles” Nanomedicine. 5(6):843-853 (2010). Other methods suitable for encapsulating materials, such as nucleic acids, into synthetic nanocarriers may be used, including without limitation methods disclosed in U.S. Pat. 6,632,671 to Unger Oct. 14, 2003.

In certain embodiments, synthetic nanocarriers are prepared by a nanoprecipitation process or spray drying. Conditions used in preparing synthetic nanocarriers may be altered to yield particles of a desired size or property (e.g., hydrophobicity, hydrophilicity, external morphology, “stickiness,” shape, etc.). The method of preparing the synthetic nanocarriers and the conditions (e.g., solvent, temperature, concentration, air flow rate, etc.) used may depend on the materials to be coupled to the synthetic nanocarriers and/or the composition of the polymer matrix.

If particles prepared by any of the above methods have a size range outside of the desired range, particles can be sized, for example, using a sieve.

In embodiments, the inventive synthetic nanocarriers can be combined with other adjuvants by admixing in the same vehicle or delivery system. Such adjuvants may include, but are not limited to mineral salts, such as alum, alum combined with monphosphoryl lipid (MPL) A of Enterobacteria, such as Escherihia coli, Salmonella minnesota, Salmonella typhimurium, or Shigella flexneri or specifically with MPL® (AS04), MPL A of above-mentioned bacteria separately, saponins, such as QS-21,Quil-A, ISCOMs, ISCOMATRIX™, emulsions such as MF59™, Montanide® ISA 51 and ISA 720, AS02 (QS21+squalene+ MPL®) , liposomes and liposomal formulations such as AS01, synthesized or specifically prepared microparticles and microcarriers such as bacteria-derived outer barrier vesicles (OMV) of N. gonorrheae, Chlamydia trachomatis and others, or chitosan particles, depot-forming agents, such as Pluronic® block co-polymers, specifically modified or prepared peptides, such as muramyl dipeptide, aminoalkyl glucosaminide 4-phosphates, such as RC529, or proteins, such as bacterial toxoids or toxin fragments. The doses of such other adjuvants can be determined using conventional dose ranging studies.

In embodiments, the inventive synthetic nanocarriers can be combined with an antigen different, similar or identical to those coupled to a nanocarrier (with or without adjuvant, utilizing or not utilizing another delivery vehicle) administered separately at a different time-point and/or at a different body location and/or by a different immunization route or with another antigen and/or adjuvant-carrying synthetic nanocarrier administered separately at a different time-point and/or at a different body location and/or by a different immunization route.

Various synthetic nanocarriers may be combined to form inventive dosage forms according to the present invention using traditional pharmaceutical mixing methods. These include liquid-liquid mixing in which two or more suspensions, each containing one or more subset of nanocarriers, are directly combined or are brought together via one or more vessels containing diluent. As synthetic nanocarriers may also be produced or stored in a powder form, dry powder-powder mixing could be performed as could the re-suspension of two or more powders in a common media. Depending on the properties of the nanocarriers and their interaction potentials, there may be advantages conferred to one or another route of mixing.

In embodiments, dosage forms according to the invention comprise inventive synthetic nanocarriers in combination with pharmaceutically acceptable excipients. The compositions may be made using conventional pharmaceutical manufacturing and compounding techniques to arrive at useful dosage forms. Techniques suitable for use in practicing the present invention may be found in Handbook of Industrial Mixing: Science and Practice, Edited by Edward L. Paul, Victor A. Atiemo-Obeng, and Suzanne M. Kresta, 2004 John Wiley & Sons, Inc.; and Pharmaceutics: The Science of Dosage Form Design, 2nd Ed. Edited by M. E. Auten, 2001, Churchill Livingstone. In an embodiment, inventive synthetic nanocarriers are suspended in sterile saline solution for injection together with a preservative. In embodiments, inventive dosage forms can comprise excipients, such as but not limited to, inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). In particular embodiments, the dosage forms may also comprise osmotic adjustment agents (e.g., salts or sugars) that are used to modify the osmolality of the dosage form to be within desired ranges (e.g. 200-500 mOsm/kg).

Inventive synthetic nanocarriers, and inventive dosage forms comprising such synthetic nanocarriers, can be used in a wide variety of applications, including delivery of osmotically active agents to desired compartments in a subject. In certain embodiments, the inventive synthetic nanocarriers can be used to deliver osmotically active agents such as isolated nucleic acids at much higher loadings than would be achievable conventionally. This characteristic can be valuable, for instance, in increasing adjuvant loadings in the synthetic nanocarriers in embodiments wherein the osmotically active agent comprises an adjuvant. The use of the inventive synthetic nanocarriers provides an additional benefit in providing more control over release rates of the osmotically active agent as compared to conventional techniques (diffusive barriers, condensing agents, etc.) for loading osmotically active agents into nanoparticles, liposomes, etc.

It is to be understood that the compositions of the invention can be made in any suitable manner, and the invention is in no way limited to compositions that can be produced using the methods described herein. Selection of an appropriate method may require attention to the properties of the osmotically active agent, the synthetic nanocarriers, and other elements of the inventive dosage forms.

In some embodiments, inventive synthetic nanocarriers are manufactured under sterile conditions or are terminally sterilized. This can ensure that resulting composition are sterile and non-infectious, thus improving safety when compared to non-sterile compositions. This provides a valuable safety measure, especially when subjects receiving synthetic nanocarriers have immune defects, are suffering from infection, and/or are susceptible to infection. In some embodiments, inventive synthetic nanocarriers may be lyophilized and stored in suspension or as lyophilized powder depending on the formulation strategy for extended periods without losing activity.

The inventive compositions may be administered by a variety of routes of administration, including but not limited to subcutaneous, intramuscular, intradermal, oral, intranasal, transmucosal, sublingual, rectal, ophthalmic, transdermal, transcutaneous or by a combination of these routes.

Doses of dosage forms contain varying amounts of synthetic nanocarriers, according to the invention. The amount of synthetic nanocarriers present in the inventive dosage forms can be varied according to the therapeutic benefit to be accomplished, and other such parameters. In embodiments, dose ranging studies can be conducted to establish optimal therapeutic amount of the synthetic nanocarriers to be present in the dosage form. Inventive dosage forms may be administered at a variety of frequencies. In a preferred embodiment, at least one administration of the dosage form is sufficient to generate a pharmacologically relevant response. In more preferred embodiment, at least two administrations, at least three administrations, or at least four administrations, of the dosage form are utilized to ensure a pharmacologically relevant response.

The compositions and methods described herein can be used to induce, enhance, suppress, modulate, direct, or redirect an immune response. The compositions and methods described herein can be used in the diagnosis, prophylaxis and/or treatment of conditions such as cancers, infectious diseases, metabolic diseases, degenerative diseases, autoimmune diseases, inflammatory diseases, immunological diseases, or other disorders and/or conditions. The compositions and methods described herein can also be used for the prophylaxis or treatment of an addiction, such as an addiction to nicotine or a narcotic. The compositions and methods described herein can also be used for the prophylaxis and/or treatment of a condition resulting from the exposure to a toxin, hazardous substance, environmental toxin, or other harmful agent.

Also within the scope of the invention are kits comprising the compositions or dosage forms of the invention with or without instructions for use and/or mixing. The kits can further contain at least one additional reagent, such as a reconstitution agent or pharmaceutically acceptable carrier, or one or more additional compositions or dosage forms of the invention. Kits containing the compositions or dosage forms of the invention can be prepared for the therapeutic applications described above. The components of the kits can be packaged either in aqueous medium or in lyophilized form. A kit may comprise a carrier being compartmentalized to receive in close confinement therein one or more container means or series of container means such as test tubes, vials, flasks, bottles, syringes, or the like. A first of said container means or series of container means may contain one or more compositions or dosage forms of the invention. A second container means or series of container means may contain an additional reagent, such as a reconstitution agent or pharmaceutically acceptable carrier.

### E. Examples

The invention will be more readily understood by reference to the following examples, which are included merely for purposes of illustration of certain aspects and embodiments of the present invention and not as limitations.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein.

Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

### Example 1: Osmolality Effect of the Outer Aqueous Phase in a W₁/O/W₂ Emulsion Used to Produce Immunostimulatory Oligonucleotide-Loaded Synthetic Nanocarriers

Dosage forms comprising osmotic mediated release barrier-free synthetic nanocarriers comprising an encapsulated osmotically active agent were prepared. In this example, the synthetic nanocarriers comprised PLGA, PLA-PEG-Nic, and PS-1826 CpG. The synthetic nanocarriers were prepared via a double emulsion method wherein the PS-1826 oligonucleotide (the osmotically active agent) was encapsulated in the nanocarriers.

Formulation elements:
W₁= 100 mg/mL of PO-1826 oligonucleotide in water, calculated osmolality = 330 mOsm/kg
W₂ = a. 5% PVA in 100 mM Phosphate buffer pH 8, calculated osmolality = 296 mOsm/kg or
b. 5% PVA in endotoxin-free RO-water, calculated osmolality = 3 mOsm/kg or
c. 5% PVA in 100 mM phosphate buffer pH 8 with 0.5 M NaCl, calculated osmolality = 1300 mOsm/kg

The polyvinyl alcohol (Mw = 11 KD - 31 KD, 87-89% partially hydrolyzed) was purchased from JT Baker. PS-1826 CpG was obtained from Oligos Etc. 9775 SW Commerce Circle C-6, Wilsonville, OR 97070). PLGA 7525 DLG 7A was purchased from from SurModics Pharmaceuticals (756 Tom Martin Drive, Birmingham, AL 35211). PLA-PEG-Nic with approximate molecular weight of 22 kD was synthesized and purified.

The above materials were used to prepare the following solutions:
1. PS-1826 CpG in water @ 100 mg/mL
2. PLGA 7525 DLG 7A in dichloromethane @ 100 mg/mL
3. PLA-PEG-Nic in dichloromethane @ 100 mg/mL
4. Polyvinyl alcohol @ 50 mg/mL in aqueous media
   Solution 1: PS-1826 CpG in aqueous solution was prepared by first dissolving PS-1826 into sterile, deionized, RNase/DNase-free water to final concentration of 100 mg/mL.
   Solution 2: PLGA 7525 DLG 7A @ 100 mg/mL in dichloromethane was prepared at room temperature and filtered with a 0.2 micron PTFE syringe filter.
   Solution 3: PLA-PEG-Nic @ 100 mg/mL in dichloromethane was prepared at room temperature and filtered with a 0.2 micron PTFE syringe filter.
   Solution 4: Polyvinyl alcohol @ 50 mg/mL was prepared in various aqueous media. Depending on the specific nanocarrier, the aqueous medium was either (a) 100 mM phosphate buffer pH 8, (b) purified water, or (c) 100 mM phosphate buffer pH 8 with 0.5 M NaCl.

A primary (W1/O) emulsion was created using Solutions 1, 2, and 3. Solution 1 (0.1 mL) was added to 1 mL of a solution containing a 3:1 v:v ratio of Solution 2 (0.75 mL) and Solution 3 (0.25 mL) in a small glass pressure tube. The primary emulsion was formed by sonicating at 50% amplitude for 40 seconds using a Branson Digital Sonifier 250.

The secondary (W1/O/W2) emulsion was then formed by adding Solution 4 (3.0 mL) to the primary emulsion and sonicating at 30% amplitude for 60 seconds using the Branson Digital Sonifier 250.

The secondary emulsion was added to a stirring beaker containing 30 mL of an aqueous Solvent Evaportion (SE) medium. Depending on the specific nanocarrier, the medium was either (a and b) 70 mM phosphate buffer pH 8 or (c) 70 mM phosphate buffer pH 8 with 0.5 M NaCl. The suspension was stirred at room temperature for 2 hours to allow the dichloromethane to evaporate and for the nanocarriers to form. A portion of the nanocarriers was washed by transferring the nanocarrier suspension to a centrifuge tube and spinning at 18,000 rcf for 60 minutes, removing the supernatant, and re-suspending the pellet in phosphate buffered saline. This washing procedure was repeated and then the pellet was dispersed and re-suspended a final time in phosphate buffered saline for a final nanocarrier dispersion with nominal concentration of 10 mg/mL on a polymer basis.

The total dry-nanocarrier mass per mL of suspension was determined by a gravimetric method. The nanocarrier entrapped PS-1826 CpG loading (%w/w) and free PS-1826 content was determined by HPLC prior to washing and again after processing was complete. Mean effective particle size was determined by DLS.

The nanocarriers were produced in similar yields (91-98%) and similar mean effective diameter sizes (230-260 nm).

Table 1

| Nanocarrier Lot | W1, W2, PBS Osmolality (mOsm/kg) | Unwashed PS-1826 Content | | Washed PS-1826 Content | |
|---|---|---|---|---|---|
| | | Entrapped (% w/w) | Free (% w/w) | Entrapped (% w/w) | Free (% w/w) |
| Lot X | 330, 296, 276 | 6.1 | 3.7 | 6.8 | 0.1 |
| Lot Y | 330, 3, 276 | 5.0 | 5.7 | 5.8 | 0.0 |
| Lot Z | 330, 1300, 276 | 7.3 | 2.8 | 6.9 | 0.9 |

Nanocarrier Lots X and Z were formed by a process that maintained a balanced near-physiologic osmolality (Lot X) or a transiently-elevated external phase osmolality (Lot Z) through to final dosage form. These nanocarriers had higher intermediate and final loadings of the osmotic agent PS-1826 than the third nanocarrier lot (Lot Y) which had been formed with a low-osmolality W2 phase. Nanocarrier Lot Z is additionally characterized by the presence of significant free osmotically-active agent PS-1826, in the final dosage form. Forming the emulsion in a hypotonic outer media led to lower encapsulation. Creating a hypertonic external medium temporarily during processing led to transiently higher loads in the particle. Once, however, the hypertonic media was replaced with isotonic media, the apparent advantage of hypertonicity was eliminated because the osmotic pressure gradient could not be effectively sustained

### Example 2: Burst Studies

The nanocarriers of Example 1 were further evaluated for burst loss of entrapped PS-1826 CpG upon a cycle of freeze and thaw.

Method of freeze-thaw cycling:

0.5 mL aliquots of the nanocarrier suspensions at approximately 7 mg nanocarrier/mL from Example 1 were shelf-frozen at -20C in 1.7 mL polypropylene centrifuge tubes. After overnight storage at -20C, the aliquots quickly transferred into a recirculating room-temperature water bath. The closed tubes were partially immersed in the in the stirred water bath such that the frozen portion in the tubes was fully below the water level. All the samples thawed within a few minutes but the aliquots were held in the bath for 20 minutes before removal for prompt analysis of particle and supernatant analysis. As in Example 1, an HPLC-based content assay was performed to determine the nanocarrier-loaded and free PS-1826 content.

Table 2

| Nanocarrier | Theoretical W1, W2, PBS Osmolality (mOsm/kg) | Washed PS-1826 Content | | Post-Freeze/Thaw Content | |
|---|---|---|---|---|---|
| | | Entrapped (% w/w) | Free (% w/w) | Entrapped (% w/w) | Free (% w/w) |
| Lot X | 330, 296, 276 | 6.8 | 0.1 | 5.1 | 1.6 |
| Lot Y | 330, 3, 276 | 5.8 | 0.0 | 3.9 | 1.3 |
| Lot Z | 330, 1300, 276 | 6.9 | 0.9 | 6.9 | 0.7 |

Nanocarriers processed and finished in an isoosmotic system led to higher entrapment levels and resulted in reduced loss of content upon freeze and thaw. Some nanocarriers demonstrated 23% loss of entrapped PS-1826 to the media whereas others exhibited 0% loss. However, when the latter nanocarriers were subsequently pelleted and transferred into fresh PBS buffer a 25% burst loss of oligonucleotide was observed. These data show the effect of a hypertonic medium in the external phase had only transient benefit and would not be helpful in the practice of the present invention due to potential side effects associated with administration of hypertonic dosage forms.

### Example 3: Low Osmolality Suspension Media can Drive Loss of Immunostimulatory Oligonucleotide from Synthetic Nanocarriers

Inventive osmotic mediated release barrier-free synthetic nanocarrier preparations were transferred (pelleted, resuspended) in various media to examine loading stability through a freeze-thaw event.

To investigate the impact of various ionic media on the freeze/thaw stability of PS-1826 CpG containing nanocarriers, the following study was performed.

Inventive nanocarriers were made according to the method of Example 1, except that Solutions 2 & 3 were replaced with a single solution containing 100 mg/mL of PLGA-PEG-Nicotine in dichloromethane. The PLGA-PEG-Nicotine was synthesized and purified and had an approximate molecular weight of 80 kD.

To transfer the nanocarriers to new media, aliquots of nanocarrier were pelleted by centrifugation (14,000 rcf, 4C), the supernant was drawn off, replaced with an equal volume of new media, and the nanocarriers were resuspended. The process was performed twice on each aliquot.

Retention of PS-1826 CpG during a freeze-thaw cycle was tested by shelf-freezing the aliquots at -20C in polypropylene centrifuge tubes, and then thawing by partial immersion in a stirred room-temperature water bath. The thawed materials were then analyzed by HPLC for free and pellet-loaded PS-1826 content. The free PS-1826 represents loss of the entrapped osmotically-active agent from the nanocarrier. The buffers, calculated osmolality, and PS-1826 content and losses are tabulated below.

Table 3

| Media* | Osmolality (mOsm/kg) | Lost PS-1826 (ug/ml) | Retained PS-1826 (ug/ml) | Loss (%) |
|---|---|---|---|---|
| Isotonic saline (0.9% NaCl) | 300 | 41.0 | 174.6 | 19 |
| 10 mM Potassium Phosphate | 29.6 | 60.7 | 156.9 | 28 |
| 10 mM Ammonium Bicarbonate | 21.9 | 58.0 | 154.7 | 27 |
| 10 mM Sodium Acetate | 20.0 | 68.4 | 150.1 | 31 |
| 10 mM Sodium Carbonate | 18.4 | 78.3 | 150.5 | 34 |
| 10 mM Glycine | 10.1 | 78.5 | 145.2 | 35 |

| | | | | |
|---|---|---|---|---|
| Endotoxin-free water | 0 | 87.1 | 136.6 | 39 |

*Adjusted with counter-ion to pH of 7-8 before use.

The results did not trend with ionic species, but loss was greater for media with lower osmolality.

### Example 4: Release Rate of Immunostimulatory Oligonucleotide can be Modulated by Osmolality of the Suspension Media

Inventive osmotic mediated release barrier-free synthetic nanocarriers were made at near-physiologic osmolality were transferred into various media at near neutral pH. The resulting release profile were controlled by the osmolality of the media. Media at isotonic condition did not lead to release.

### Materials

PO-1826 DNA oligonucleotide with phosphodiester backbone having nucleotide sequence 5‘-TCC ATG ACG TTC CTG ACG TT-3‘ (SEQ ID NO: 1) with a sodium counter-ion was purchased from Oligo Factory (120 Jeffrey Ave., Holliston, MA 01746.)

PLA with an inherent viscosity of 0.21 dL/g was purchased from SurModics Pharmaceuticals (756 Tom Martin Drive, Birmingham, AL 35211. Product Code 100 DL 2A.)

PLA-PEG-Nicotine with a molecular weight of approximately 22,000 Da was synthesized using conventional methods.

Polyvinyl alcohol (Mw = 11,000 - 31,000, 87-89% hydrolyzed) was purchased from J.T. Baker (Part Number U232-08).
Solution 1: PO-1826 CpG in aqueous solution was prepared by first dissolving PO-1826 into sterile, deionized, RNase/DNase-free water to a concentration of 40 mg/mL.
Solution 2: PLA @ 75 mg/mL and PLA-PEG-nicotine @ 25 mg/ml in dichloromethane. The solution was prepared by combining two separate solutions at room temperature: PLA in dichloromethane and PLA-PEG-nicotine in dichloromethane, each filtered with a 0.2 micron PTFE syringe filter. The final solution was prepared by adding 3 parts PLA solution for each part of PLA-PEG-nicotine solution.
Solution 3: Polyvinyl alcohol @ 50 mg/mL in 100 mM pH 8 phosphate buffer. Solution 4: 70 mM phosphate buffer pH 8

A primary (W1/O) emulsion was created using Solution 1 & Solution 2. Solution 1 (0.25 mL) and Solution 2 (1.0 mL) were combined in a small glass pressure tube and sonicated at 50% amplitude for 40 seconds using a Branson Digital Sonifier 250.

The secondary (W1/O/W2) emulsion was then formed by adding Solution 3 (3.0 mL) to the primary emulsion and sonicating at 30% amplitude for 60 seconds using the Branson Digital Sonifier 250.

The second emulsion was added to a beaker containing Solution 4 (30 mL) and stirred at room temperature for 2 hours to allow for the dichloromethane to evaporate and for the nanocarriers to form. A portion of the nanocarriers were washed by transferring the nanocarrier suspension to a centrifuge tube and spinning at 21,000 rcf for 45 minutes, removing the supernatant, and re-suspending the pellet in phosphate buffered saline. This washing procedure was repeated and then the pellet was re-suspended in phosphate buffered saline for a final nanocarrier dispersion with nominal concentration of 10 mg/mL on a polymer basis.

The total dry-nanocarrier mass per mL of suspension was determined by a gravimetric method. The PO-1826 CpG content of in the nanocarrier was determined by HPLC.

The in vitro release (IVR) rate in various media was determined by centrifugal pelleting an aliquot of the nanocarrier and withdrawing the supernatant, resuspending the nanocarrier the new media, and incubating with agitation at 37C for 24 hours. PO-1826 CpG release was determined by HPLC at time of resuspension (t = 0 hours), 2 hours, 6 hours, and at 24 hours in the release media. Release was calculated as a percentage. The release media, burst release at time 0, and release over 24 hours is tabulated and graphed below.

Table 4

| Release Media & pH | Calculated Osmolality (mOsm/kg) | Burst Release (%) | 24 hour Release (%) |
|---|---|---|---|
| 10 mM Phosphate + 150 mM NaCl, pH 7.35 | 328 | 2 | 3 |
| 100 mM Phosphate, pH 7.5 | 275 | 18 | 25 |
| 10 mM Phosphate + 50 mM | 225 | 23 | 21 |
| 10 mM Phosphate + 50 mM NaCl, pH 7.35 | 128 | 22 | 32 |
| 10 mM Phosphate, pH 7.35 | 28 | 61 | 63 |

Osmotic control of release is observed at physiologic pH (pH 7-8). As shown in the figure and table above, particles suspended in low-osmolality media (e.g., 28 mOsm/kg) quickly release entrapped active osmotic agent in significant amounts. As media with increasingly higher osmolality are used (with either NaCl, sodium phosphate, and/or EDTA used to establish osmolality), the percent release at T = 0 h and 24 h is reduced correspondingly. Near-zero release of the osmotic agent into media of physiologic-osmolality and physiologic pH indicates the stability of the nanocarrier in a preparation suitable for administration.

### Example 5: Nicotine Vaccination Experiments

Osmotic-mediated release synthetic nanocarriers may be formulated with sensitivity to pH at near-physiologic osmolality. The release rate of the active osmotic agent as a function of pH may relate to the potency of pharmacologic effect. The objectives of the two experiments detailed below were twofold: (1) to confirm that more potent nanocarriers were achieved with the same nanocarrier materials and formation methods when the selection of media was designed to not expose the nanocarriers to prolonged osmotic gradients of greater than approximately 140 mOsm/kg (calculated as nanocarrier-phase osmolality minus average system osmolality including suspension media) and (2) to evaluate the relationship between in-vitro release rates in acidic media of a CpG adjuvant from nanocarriers to their potency. Potency in both cases is measured in terms of the levels of of antibodies induced by the adjuvant-loaded antigen-presenting nanocarriers.

### Nanoparticle Formulation and IVR Determination

### Materials

PO-1826 DNA oligonucleotide with phosphodiester backbone having nucleotide sequence 5‘-TCC ATG ACG TTC CTG ACG TT-3‘ (SEQ ID NO: 1) with a sodium counter-ion was purchased from Oligo Factory (120 Jeffrey Ave., Holliston, MA 01746.) Ovalbumin peptide 323-339, a 17 amino acid peptide known to be a T and B cell epitope of Ovalbumin protein, was purchased from Bachem Americas Inc. (3132 Kashiwa Street, Torrance CA 90505. Part # 4065609.) PLA with an inherent viscosity of 0.21 dL/g was purchased from SurModics Pharmaceuticals (756 Tom Martin Drive, Birmingham, AL 35211. Product Code 100 DL 2A.)

PLGA with varied inherent viscosities (IV) and lactide:glycolide (L:G) ratios were purchased from SurModics Pharmaceuticals (756 Tom Martin Drive, Birmingham, AL 35211.) or Boehringer Ingelheim (55216 Ingelheim am Rhein, Germany). The product codes, manufacturer, IV, and L:G ratios were as tabulated below.

Table 5

| Product code | Manufacturer | IV (dL/g) | L:G ratio |
|---|---|---|---|
| 5050 DLG 2.5A | Surmodics | 0.25 | 52:48 |
| RG653H | Boehringer Ingelheim | 0.3 | 65:35 |
| 7525 DLG 7A | Surmodics | 0.75 | 75:25 |

PLA-PEG-Nicotine with a molecular weight of approximately 22,000 Da was synthesized using conventional methods. Polyvinyl alcohol (Mw = 11,000 - 31,000, 87-89% hydrolyzed) was purchased from J.T. Baker (Part Number U232-08).

### Method for Synthetic Nanocarrier Lot A (MHC II Peptide Nanocarrier)

Solution 1: Ovalbumin peptide 323 - 339 @ 40 mg/mL in 0.13 N hydrochloric acid (HCl). The solution was prepared by dissolving ovalbumin peptide directly in 0.13 N HCl solution at room temperature and then filtering with a 0.2 micron PES syringe filter.

Solution 2: 0.21-IV PLA @ 75 mg/mL and PLA-PEG-nicotine @ 25 mg/ml in dichloromethane. The solution was prepared by first making two separate solutions at room temperature: 0.21-IV PLA @ 100 mg/mL in pure dichloromethane and PLA-PEG-nicotine @ 100 mg/mL in pure dichloromethane, each filtered with a 0.2 micron PTFE syringe filter. The final solution was prepared by adding 3 parts PLA solution for each part of PLA-PEG-nicotine solution.

Solution 3: Polyvinyl alcohol @ 50 mg/mL in 100 mM pH 8 phosphate buffer.

Solution 4: 70 mM phosphate buffer pH 8

A primary (W1/O) emulsion was created using Solution 1 & Solution 2. Solution 1 (0.2 mL) and Solution 2 (1.0 mL) were combined in a small glass pressure tube and sonicated at 50% amplitude for 40 seconds using a Branson Digital Sonifier 250. The secondary (W1/O/W2) emulsion was then formed by adding Solution 3 (3.0 mL) to the primary emulsion and sonicating at 30% amplitude for 60 seconds using the Branson Digital Sonifier 250.

The second emulsion was added to a beaker containing 70 mM phosphate buffer solution (30 mL) and stirred at room temperature for 2 hours to allow for the dichloromethane to evaporate and for the nanocarriers to form. A portion of the nanocarriers were washed by transferring the nanocarrier suspension to a centrifuge tube and spinning at 21,000 rcf for 45 minutes, removing the supernatant, and re-suspending the pellet in phosphate buffered saline. This washing procedure was repeated and then the pellet was re-suspended in phosphate buffered saline for a final nanocarrier dispersion with nominal concentration of 10 mg/mL on a polymer basis.

The total dry-nanocarrier mass per mL of suspension was determined by a gravimetric method. The peptide content of the nanocarrier was determined by HPLC to be 4.1% w/w. The nanocarrier concentration was diluted to 5 mg/mL before use by adding phosphate buffered saline.

### Method for Nanocarrier lots B, C, D, E, F, & G (CpG-Containing Nanocarriers)

Solution 1: PO-1826 CpG in aqueous solution was prepared by first dissolving PO-1826 into sterile, deionized, RNase/DNase-free water to make a concentrated stock solution (e.g., 200 mg/mL). The solution was diluted to 40 mg/mL with either additional water or with an aqueous KCl solution. The final solution 1 media used to make each synthetic nanocarrier lot are tabulated below.

Table 6

| Nanocarrier Lot | Solution 1 medium | Solution 1 Calculated Osmolality (mOsm/kg) |
|---|---|---|
| B | 150 mM KCl | 432 |
| C | Water | 132 |
| D | Water | 132 |
| E | Water | 132 |
| F | 125 mM KCl | 382 |
| G | 125 mM KCl | 382 |
| H | 150 mM KCl | 432 |

Solution 2: PLGA @ 75 mg/mL and PLA-PEG-nicotine @ 25 mg/ml in dichloromethane. The solution was prepared by combining two separate solutions at room temperature: PLGA in dichloromethane and PLA-PEG-nicotine in dichloromethane, each filtered with a 0.2 micron PTFE syringe filter. The final solution was prepared by adding 3 parts PLA solution for each part of PLA-PEG-nicotine solution. The PLGA composition used to prepare each nanocarrier is tabulated below. In the case of Lot E, the dichloromethane additional included 5% v/v benzyl alcohol, which was found to reduce PO-1826 entrapment efficiency yet maintain an intermediate rate of PO-1826 release.

Table 7

| | |
|---|---|
| Nanocarrier Lot | PLGA Source |
| B | 7525 DLG 7A |
| C | 7525 DLG 7A : 5050 DLG 2.5A @ 2:1 weight ratio |
| D | 7525 DLG 7A |
| E | 7525 DLG 7A |
| F | RG653H |
| G | 7525 DLG 7A |
| H | 7525 DLG 7A : 5050 DLG 2.5A @ 2:1 weight ratio |

Solution 3: Polyvinyl alcohol @ 50 mg/mL in 100 mM pH 8 phosphate buffer (calculated solution osmolality 298 mOsm/kg). In the case of Lot D the phosphate buffer was replaced with 150 mM KCl (calculated solution osmolality 304 mOsm/kg).

Solution 4: 70 mM phosphate buffer pH 8 (calculated solution osmolality 206 mOsm/kg). In the case of S0890-09-7 solution 4 was purified water (effectively zero osmolality).

A primary (W1/O) emulsion was created using Solution 1 & Solution 2. Solution 1 (0.25 mL) and Solution 2 (1.0 mL) were combined in a small glass pressure tube and sonicated at 50% amplitude for 40 seconds using a Branson Digital Sonifier 250.

The secondary (W1/O/W2) emulsion was then formed by adding Solution 3 (3.0 mL) to the primary emulsion and sonicating at 30% amplitude for 60 seconds using the Branson Digital Sonifier 250.

The second emulsion was added to a beaker containing Solution 4 (30 mL) and stirred at room temperature for 2 hours to allow for the dichloromethane to evaporate and for the synthetic nanocarriers to form. A portion of the synthetic nanocarriers were washed by transferring the synthetic nanocarrier suspension to a centrifuge tube and spinning at 21,000 rcf for 45 minutes, removing the supernatant, and re-suspending the pellet in fresh

Solution 4. This washing procedure was repeated and then the pellet was re-suspended in phosphate buffered saline for a final synthetic nanocarrier dispersion with nominal concentration of 10 mg/mL on a polymer basis.

The total dry synthetic nanocarrier mass per mL of suspension was determined by a gravimetric method. The PO-1826 CpG content of the synthetic nanocarriers was determined by HPLC. The synthetic nanocarrier concentration was diluted to 5 mg/mL before use by adding phosphate buffered saline.

The in vitro release (IVR) rate was determined by centrifugal pelleting an aliquot of the synthetic nanocarriers, resuspending the synthetic nanocarriers in 100 mM pH 4.5 citrate buffer, and incubating with agitation at 37C for 24 hours. PO-1826 CpG release was determined by HPLC at time of resuspension (t = 0 hours), at 6 hours, and at 24 hours in the release media. The IVR was calculated by subtracting the t0 release from the 24-hour release, and normalizing per synthetic nanocarrier mass. PO-1826 CpG load and IVR (24h-0h) for the synthetic nanocarriers is tabulated below.

Table 8

| Nanocarrier Lot | Max. outward-directed osmotic gradient (mOsm/kg) | PO-1826 Load (% w/w) | IVR (24 h-0 h) ug-CpG/mg-NC |
|---|---|---|---|
| B | 134 | 7.5 | 13 |
| C | 79 | 7.0 | 22 |
| D | 291 | 4.6 | 3 |
| E | 79 | 4.9 | 9 |
| F | 98 | 9.0 | 31 |
| G | 98 | 8.8 | 18 |
| H | 134 | 6.6 | 25 |

Nanocarrier D demonstrates the load-reducing impact of processing with a high outward-directed osmotic gradient resulting from the use of purified water as the solvent-evaporation medium having osmolality significantly less than 200 mOsm/kg. The 4.6% load of CpG in nanocarrier D is reduced compared in particular to nanocarriers B and G, which have the same polymeric composition. The reduced loading of nanocarrier D is also associated with a reduced IVR as measured in acidic medium.

### Vaccination

Naive C57BL/6 female mice, 5 animals per nanoparticle group, were inoculated with nicotine vaccine nanoparticles. Inoculations were made subcutaneously into the hind pads of naive C57BL/6 females (5 animals per group) according to a schedule of a prime on day 0 followed by boosts on days 14 and 28. For each inoculation a total of 100 µg nanocarrier (NC) was injected, divided equally between the hind limbs. Planned sera collection and analysis for anti-nicotine antibody titers were performed at days 26 and 40. Anti-nicotine IgG antibody titers were measured by ELISA and are reported as EC50 values.

Each animal received inoculations that contained a 1:1 mixture of two different nanocarriers; one providing an MHC II peptide (Lot A), a second providing a CpG adjuvant (Lots B-G). Both particles presented nicotine. The same lot of MHC II peptide-containing nanocarrier, Lot A, was used in all groups. The CpG-containing nanocarrier was different for each group (i.e. different lots were used). The CpG-containing nanocarriers differed in their PLGA composition and CpG loading, leading to different in vitro release (IVR) rates of CpG into an acidic medium. In the case of nanocarrier E, the release rate was also affected by the use of benzyl alcohol in the nanocarrier formation process.

The CpG nanocarrier and IVR are presented for each group along with the resulting anti-nicotine antibody titer (mean EC50 and standard deviation) at day 40 (Tables 9 & 2=10).

Study 1 directly compared the potency of the CpG-containing nanocarrier lots B, C, D, and E. As tabulated below, there was a direct relationship between the release rate in acidic medium and the resulting peak (day 40) titers.

Table 9

| CpG Nanocarrier | Net 24 h IVR (µg/mg-NP) | Anti-Nicotine Antibody Titer (EC₅₀) |
|---|---|---|
| C | 22 | 891,000 |
| B | 13 | 278,000 |
| E | 9 | 260,000 |
| D | 3 | 99,000 |

Three of the four nanocarriers of the above example were prepared with control of osmotic gradients to limit CpG losses during processing and storage. Nanocarrier group D had reduced load and IVR due to the significant gradient introduced during a processing step, and the impact can be seen in the potency of anti-nicotine antibody generation. While nanocarriers of groups B and D were made of the same materials, vaccination with the group D nanocarriers resulted in approximately ⅓ the titer generation.

Further evident in the study is the value that can be created by modulating the composition of the osmotic barrier-free synthetic nanocarriers such that pH-influence on release is adjusted. The pH triggered osmotic mediated release barrier-free synthetic nanocarriers having greater acidic sensitivity (higher acidic-IVR of the CpG adjuvant) generated higher antibody titers to the target antigen.

The relationship of increasing titer with increasing acidic-medium IVR (per the IVR protocol above) was repeated in a follow-up study (Study 2). pH triggered osmotic mediated release barrier-free synthetic CpG-containing nanocarriers lots F, H, C, and G were evaluated in a head-to-head anti-nicotine vaccination study. As with study 1, the results tabulated below demonstrate increasing in vivo potency with increasing IVR in an acidic medium.

Table 10

| CpG Nanocarrier | Net 24 h IVR (µg/mg-NP) | Anti-Nicotine Antibody Titer (EC₅₀) |
|---|---|---|
| F | 31 | 565,000 |
| H | 25 | 397,000 |
| C | 22 | 377,000 |
| G | 18 | 221,000 |

In all instances, the osmotic barrier-free nanocarriers were processed and handled to avoid outward-directed gradients that would significantly reduce the load of the entrapped osmotically-active agent, CpG. This process and formulation approach again enabled the modulation of acidic-IVR rates through polymeric composition. As with the previous example, within the range of IVR evaluated, higher rates of CpG release resulted in greater potency as evidenced by the antigen-specific antibody titers.

1. A dosage form comprising:
osmotic mediated release barrier-free synthetic nanocarriers comprising an encapsulated osmotically active agent.

2. The dosage form of claim 1, further comprising a vehicle having an osmolality of 200-500 mOsm/kg.

3. The dosage form of claim 1, wherein the osmotically active agent is present in the synthetic nanocarriers in an amount of about 2 weight percent, based on the total theoretical weight of the synthetic nanocarriers.

4-9. (canceled)

10. The dosage form of claim 1, wherein the osmotically active agent comprises an isolated nucleic acid, a polymer, an isolated peptide, an isolated saccharide, macrocycle, or ions, cofactors, coenzymes, ligands, hydrophobically-paired agents, or hydrogen-bond donors or acceptors thereof.

11. The dosage form of claim 10, wherein the isolated nucleic acid comprises: an immunostimulatory nucleic acid, immunostimulatory oligonucleotides, small interfering RNA, RNA interference oligonucleotides, RNA activating oligonucleotides, micro RNA oligonucleotides, antisense oligonucleotides, aptamers, gene therapy oligonucleotides, natural form plasmids, nonnatural plasmids, chemically modified plasmids, chimeras that include oligonucleotide-based sequences, and combinations of any of the above.

12. The dosage form of claim 10, wherein the polymer comprises osmotically active: dendrimers, polylactic acids, polyglycolic acids, poly lactic-co-glycolic acids, polycaprolactams, polyethylene glycols, polyacrylates, polymethacrylates, and co-polymers and/or combinations of any of the above.

13. The dosage form of claim 10, wherein the isolated peptide comprises osmotically active: immunomodulatory peptides, MHC Class I or MHC Class II binding peptides, antigenic peptides, hormones and hormone mimetics, ligands, antibacterial and antimicrobial peptides, anticoagulation peptides, and enzyme inhibitors.

14. The dosage form of claim 10, wherein the isolated saccharide comprises osmotically active: antigenic saccharides, lipopolysaccharides, protein or peptide mimetic saccharides, cell surface targeting saccharides, anticoagulants, anti-inflammatory saccharides, antiproliferative saccharides, including their natural and modified forms, monosaccharides, disaccharides, trisaccharides, oligosaccharides, or polysaccharides.

15. The dosage form of claim 1, wherein the osmotic mediated release barrier-free synthetic nanocarriers comprise pH triggered osmotic mediated release barrier-free synthetic nanocarriers.

16. A method comprising:
forming osmotic mediated release barrier-free synthetic nanocarriers that comprise an osmotically active agent in an environment having an osmolality ranging from 200-500 mOsm/kg; and
maintaining the formed osmotic mediated release barrier-free synthetic nanocarriers in an environment having an osmolality ranging from 200-500 mOsm/kg.

17. The method of claim 16, wherein the environment in which the osmotic mediated release barrier-free synthetic nanocarriers are formed, and the environment in which the osmotic mediated release barrier-free synthetic nanocarriers are maintained, are the same.

18. The method of claim 16, further comprising:
processing the formed osmotic mediated release barrier-free synthetic nanocarriers in an environment having an osmolality ranging from 200-500 mOsm/kg.

19. The method of claim 18, wherein processing comprises: washing the synthetic nanocarriers, centrifuging the synthetic nanocarriers, filtering the synthetic nanocarriers, concentrating or diluting the synthetic nanocarriers, freezing the synthetic nanocarriers, drying the synthetic nanocarriers, combining the synthetic nanocarriers with other synthetic nanocarriers or with additive agents or excipients, adjusting the pH or buffer environment of the synthetic nanocarriers, entrapping the synthetic nanocarriers in a gel or high-viscosity medium, resuspending the synthetic nanocarriers, surface modifying the synthetic nanocarriers covalently or by physical processes such as coating or annealing, impregnating or doping the synthetic nanocarriers with active agents or excipients, sterilizing the synthetic nanocarriers, reconstituting the synthetic nanocarriers for administration, or combinations of any of the above.

20. The method of claim 16, further comprising storing the formed osmotic mediated release barrier-free synthetic nanocarriers in an environment having an osmolality ranging from 200-500 mOsm/kg.

21. The method of claim 16, further comprising formulating the formed osmotic mediated release barrier-free synthetic nanocarriers into a dosage form that maintains the formed osmotic mediated release barrier-free synthetic nanocarriers in an environment having an osmolality ranging from 200-500 mOsm/kg.

22. The method of claim 16, wherein the osmotically active agent is present in the synthetic nanocarriers in an amount of about 2 weight percent, based on the total theoretical weight of the synthetic nanocarriers.

23-33. (canceled)

34. A process for producing a dosage form comprising osmotic mediated release barrier-free synthetic nanocarriers comprising the method steps as defined in claim 16.

35. A dosage form comprising osmotic mediated release barrier-free synthetic nanocarriers made according to any of the method of claim 16.

36. A lyophilized dosage form comprising:
lyophilized osmotic mediated release barrier-free synthetic nanocarriers comprising an encapsulated osmotically active agent; and
lyophilizing agents that provide a vehicle having an osmolality of 200-500 mOsm/kg upon reconstitution of the lyophilized dosage form.

37-51. (canceled)

52. A method comprising:
providing osmotic mediated release barrier-free synthetic nanocarriers that comprise an osmotically active agent in an environment having an osmolality ranging from 200-500 mOsm/kg; and
administering the osmotic mediated release barrier-free synthetic nanocarriers to a subject.

53-68. (canceled)

69. A method of administering the dosage form of claims 1 to a subject in need thereof.

70-71. (canceled)

72. A kit, comprising the dosage form of claim 1.

73-80. (canceled)


SEQUENCE LISTING (1 sequence)

SEQ ID NO: 1 - DNA, 20 bp, synthetic construct
  source 1..20: mol_type = other DNA
tccatgacgttcctgacgtt